(19) United States
(12) Patent Application Publication
(10) Pub. No.: US 2006/0205671 A1
(43) Pub. Date: Sep. 14, 2006

(54) Compositions and methods for use of a protease inhibitor and adenosine for preventing organ ischemia and reperfusion injury

(76) Inventor: Jakob Vinten-Johansen, Grayson, GA (US)

Correspondence Address: MORRIS MANNING & MARTIN LLP, 1600 ATLANTA FINANCIAL CENTER, 3343 PEACHTREE ROAD, NE, ATLANTA, GA, 30326-1044, US

(21) Appl. No.: 10/562,757

(22) PCT Filed: Jul. 2, 2004
(86) PCT No.: PCT/US04/21387

§ 371 Date: Mar. 28, 2006

(30) Foreign Application Priority Data
Jul. 2, 2003 (US) 60484484

(51) Int. Cl. A61K 38/05; A61K 38/04; A61K 31/7076; A61K 31/366; A61K 38/54; A61K 31/66; A61K 31/185; A61K 31/195

(52) U.S. Cl. 514/18; 514/19; 514/45; 514/46; 514/457; 514/553; 514/561; 514/75; 424/94.3

(57) ABSTRACT

Methods and compositions including combined use of a serine protease inhibitor and adenosine when administered as a single pharmaceutical composition, concomitantly or sequentially in any order to a living subject for preventing organ ischemia or reperfusion injury. The methods and compositions disclosed herein can be used in such procedures as cardiac surgery, non-surgical cardiac revascularization, organ transplantation, perfusion, ischemia, reperfusion, ischemia-reperfusion injury, oxidant injury, cytokine induced injury, shock induced injury, resuscitations injury or apoptosis.

This application is being filed as a PCT International application in the name of Emory University, a U.S. national corporation, applicant for the designation of all countries except the U.S., and by Jakob Vinten-Johansen, a U.S. national and resident, applicant for the designation of the U.S. only, on 2 Jul. 2004.

Some references, which may include patents, patent applications and various publications, are cited in a reference list and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference. In terms of notation, hereinafter, “[n]” represents the nth reference cited in the reference list. For example, [5] represents the 5th reference cited in the reference list, namely, Fernandez A Z, Williams M W, Jordan J E, Zhao Z-Q, Vinten-Johansen J., Neutrophil (PMN) adherence to throinbin stimulated coronary vascular endothelium is inhibited by an adenosine (ADO) A₂-receptor mechanism. FASEB Journal 10, A611. 1996.

### FIELD OF THE INVENTION

The present invention relates to a pharmaceutical composition comprising a protease inhibitor and adenosine and methods of using same for ischemia-reperfusion injury prevention.

### BACKGROUND OF THE INVENTION

Following exposure to a pathogenic injury or disease, vascularized tissue will initiate an inflammatory response in order to eliminate harmful agents from the body. A wide range of pathogenic insults can initiate inflammatory response including infection, allergens, autoimmune stimuli, immune response to transplanted tissue, noxious chemicals, toxins, ischemia-reperfusion, hypoxia, and mechanical and thermal trauma. Although inflammatory responses may have beneficial effects such as indicating the presence of infection or other injury that require medical attention, they may also exert harm if host tissues are damaged in the process of eliminating the diseased areas. For example, inflammation causes the pathologies associated with rheumatoid arthritis, myocardial infarction, ischemia-reperfusion injury, hypersensitivity reactions, and certain types of fatal autoimmune renal disease.

In the case of hypoxia or ischemia, constriction or obstruction of a blood vessel causes reduced blood flow and, hence, reduced oxygen to a bodily organ or tissue; reperfusion is necessary to prevent cell death from totally engulfing the area placed at risk [13, 14]. The ensuing inflammatory responses to reperfusion injury provide additional insult to the affected tissue. Examples of hypoxia or ischemia include the partial or total loss of blood supply to the body as a whole, an organ within the body, or a region within an organ, such as those that occur in cardiac arrest, pulmonary embolus, renal artery occlusion, coronary occlusion or occlusive stroke.

In the cardiovascular setting, early reperfusion salvages myocardium that would otherwise be destined to die by either necrosis or apoptosis. The salvage of myocardium by timely reperfusion is associated with lower morbidity, lower mortality, and a greater chance for return to an acceptable lifestyle for the patient. Reperfusion can be achieved in a catheterization laboratory using catheter-based technology such as percutaneous transluminal coronary angioplasty (PTCA) alone or in conjunction with deployment of stents, and adjunct intravenous delivery of thrombolytic therapy (tissue plasminogen activator tPA, urokinase, streptokinase). Nevertheless, ensuing inflammatory responses may lead to reperfusion injury. Although revascularization of acutely occluded coronary arteries is 85% to 95% successful in the catheterization laboratory, 40% of these cases result in complications arising from the reperfusion, including arrhythmias, ventricular fibrillation, contractile failure and infarction. The tissue damage associated with ischemia-reperfusion injury is believed to comprise both the initial cell damage induced by the deprivation of oxygen to the cell and its subsequent recirculation, as well as the damage caused by the body's inflammatory response to this initial damage.

The Inflammatory Component of Reperfusion Injury

The inflammation component of reperfusion injury is initiated by the interaction between polymorphonuclear neutrophils (PMNs), the chief phagocytic leukocytes, and coronary vascular endothelium. It consists of highly specific and temporally orchestrated sequence of events involving the early (P-selectin, L-selectin) and late (ICAM-1, VCAM, PECAM) expression of adhesion molecules on both endothelium and PMNs, which is further described infra in connection with FIG. 2. This interaction begins immediately upon reperfusion, and may continue for over 72 hours [15].

During the early moments of reperfusion and/or inflammation, in response to oxygen radical species, the serine protease thrombin, histamine, tumor necrosis factor-alpha (TNF□), platelet activating factor, and IL-1, the pro-adhesive properties of endothelium are stimulated [16-19]. P-selectin, stored as preformed granules in the Weibel-Palade bodies, is rapidly translocated to the endothelial surface [21-23]. Interaction with P-selectin on endothelium causes the neutrophil to start rolling and attaching loosely on the endothelial surface [17, 24]. This “rolling phenomenon” plays a critical role in the pathogenesis of the early phase of reperfusion injury in myocardium [25]. These same factors are also known stimulants of tissue factor. The endothelium may be further stimulated by thrombin generated by tissue factor localized on its cell surface, by neutrophils/monocytes circulating in the region, and by myocytes [20].

Of all the factors that stimulate inflammatory response, the serine protease thrombin is of particular importance. Preliminary observations confirm that thrombin is a potent stimulator of P-selectin expression in endothelium, and promotes neutrophil adhesion to coronary vascular endothelium. Co-incubation of neutrophils with coronary artery segments that have been activated with thrombin results in significant endothelial dysfunction that is not observed in normal segments or segments not activated with thrombin, which is further described infra in connection with FIG. 3 [8, 21, 24, 26-33]. Thrombin also stimulates platelet activation (via PAR-1 receptors), causing activated platelets to express P-selectin on their membranes.

After the initial tethering of PMNs to the vascular endothelium, firm adherence is facilitated by interaction between CD11b/CD18 on PMNs and ICAM-1 on the endothelium. ICAM-1 is constitutively expressed at low levels, but de novo protein synthesis and surface expression is stimulated by cytokines (e.g., TNF□) beginning at 4-6 hours after reperfusion, and peaking at 24 hours. Studies confirm that endothelial ICAM-1 is not significantly expressed until between 6 and 24 hours of reperfusion, with expression in myocytes occurring later than 24-72 hours [15, 34]. This later response is in contrast to the early (<30 minutes) expression of P-selectin.

Firm adhesion of PMNs to the vascular endothelium is followed by transendothelial migration of PMNs into the extravascular (myocyte) compartment. The early PMNs adherence to endothelium is prerequisite to a constellation of pathophysiological processes that ultimately lead to infarction, contractile dysfunction, microvascular injury, endothelial cell dysfunction, and apoptosis. However, the continued interaction between neutrophils and endothelium in later phases of reperfusion (6-72 hours) leads to expansion of necrosis and no-reflow zones, and the initiation of apoptosis [35]. The development of apoptosis has been reported to be triggered primarily during reperfusion, and is therefore a “reperfusion event” [36].

By administering agents that could effectively inhibit different or all phases of inflammation, the pathophysiological consequences associated with it could be minimized. Adenosine and aprotinin are two such agents whose inflammation inhibitory mechanisms of action have been extensively investigated. However, the combination of adenosine and aprotinin, and their complimentary affects in reperfusion injury, have not been investigated or used in practice.

Adenosine in Cardioprotection

Adenosine is a cardioprotective autacoid that is present in small quantities (less than 1 μM) in the normal myocardium, and is transiently increased during ischemia by sequential degradation of high-energy phosphates (ATP, ADP, and AMP). The physiological tissue levels of adenosine are regulated by the production and release of adenosine by cardiac myoctyes, the endothelium, neutrophils and other cell types. Adenosine interacts with specific G-protein coupled purinergic (adenosinergic) receptors on the endothelium, myocytes or neutrophils to elicit a wide range of physiological responses not unlike those of nitric oxide (NO). The physiologic effect resulting from activation of the specific adenosinergic receptor is transduced by either stimulating adenylate cyclase (Gₛ) and increasing cAMP levels (A₂ recepteors) or inhibiting adenylate cyclase (Gi) and decreasing cAMP levels (A₁ and A₃ receptors). The physiologically diverse effects of adenosine are related to the differential effects on the G-protein coupled receptors and post-receptor effectors such as K_{ATP} channels, protein kinase C (PKC) activity, phosphatidylinositol-3 (PI-3) kinase, nitric oxide synthase, potassium channels, and sodium-hydrogen exchange (NHE) systems to name a few. Therefore, adenosine can exert a broad spectrum of effects on key components (neutrophils, endothelium) and compartments (intravascular, interstitial, myocyte) involved in ischemia-reperfusion injury. The target of these receptor-mediated interactions has implications as to the time course of administration of therapeutics.

Adenosine is a potent inhibitor of neutrophil functions. Cronstein et al. [37] reported that adenosine inhibited superoxide generation by neutrophils activated by fMLP, A23187, and concanavalin A. Later studies determined that this inhibitory effect was mediated by the A₂ adenosine receptor [38]. Studies from our laboratory confirmed the attenuation of superoxide generation in a concentration-dependent manner by A₂ receptor mechanism [8]. Furthermore, the selective A₂ₐ agonist CGS-21680 attenuated superoxide production in a manner similar to adenosine. However, the A₃ adenosinergic receptor does not seem to regulate neutrophil superoxide anion generation [39]. In addition to directly inhibiting neutrophil respiratory burst, adhesion and degranulation, adenosine also inhibits platelet activities. Adenosine inhibits platelet aggregation in concentrations ranging from 2-40 μM exogenous adenosine. Hence, the cooperative activation between platelets and neutrophils, leading to amplified neutrophil activation during ischemia-reperfusion, may be attenuated by adenosine. The anti-platelet concentration of adenosine is well within the range (10 μM) that would be used for intracoronary therapeutics to reduce ischemia-reperfusion injury.

Prolonged coronary occlusion followed by reperfusion produces necrosis within the area at risk, beginning in the subendocardium and extending with occlusion time toward the subepicardium in a wavefront pattern. In a landmark study, Olafsson et al. [40] first reported that intracoronary adenosine, transiently infused into the LAD at 3.75 mg/min at the onset of reperfusion, reduced infarct size by 75% and improved regional contractile function 24 hours after the start of reflow. Histology demonstrated preservation of endothelial morphology with decreased neutrophil infiltration and plugging in the central necrotic zone. This study [40] is important because it demonstrated that adenosine could (a) reduce infarct size on a long term basis (inhibition versus delay) when adenosine was administered at the onset of reperfusion, thereby identifying the reperfusion period as a feasible therapeutic time point, (b) inhibit neutrophil accumulation in the area at risk, or at least attenuate plugging of the capillaries, (c) reduce endothelial damage, and (d) attenuate the complex processes of reperfusion injury leading to contractile dysfunction. These data strongly suggested an interaction between neutrophils and the vascular endothelium in the pathogenesis of infarction, which has since emerged as a key triad in the pathogenesis of reperfusion injury.

Similar results were subsequently found by others using intravenous administration of adenosine [41] or adenosine receptor-specific analogues [42-45]. The attenuation of endothelial injury with intracoronary adenosine was reinforced by subsequent studies from the same group [46, 47]. Using in vivo determination of endothelial-dependent (acetylcholine) and independent (papaverine) vasodilator reserve as a surrogate measure of endothelial function, both components of vasodilator responses were attenuated after reperfusion, consistent with the in vitro studies by Cronstein et al. [37] and Zhao et al. [8]. In addition, regional myocardial blood flow was impaired, which is consistent with microvascular injury. Adenosine attenuated the loss of vasodilator reserve, and also reduced neutrophil infiltration and morphologic injury to the endothelium. These studies, therefore, confirmed that adenosine reduces necrosis, likely by preventing neutrophil accumulation and microvascular injury.

Since adenosine has potent direct anti-neutrophil properties, it is hypothesized that adenosine would reduce reperfusion injury in part by inhibiting neutrophil events, including accumulation in the area at risk, through an A₂ receptor mechanism. Jordan et al. [6] used a canine model of LAD occlusion with reperfusion via a carotid-to-LAD shunt used to introduce pharmacologic agents intracoronarily. After 60 minutes of collateral-deficient (LAD arteriotomy) occlusion, reperfusion was initiated with an infusion of either saline (control) or the A₂ receptor specific analogue CGS-21680 for the first hour of reperfusion. Similar to the study by Schlack et al. [48], Jordan et al. found that CGS-21680 significantly reduced infarct size from 29.8±2.3% of the area at risk in a saline vehicle group to 15.4±2.9% of the area at risk. However, there was no improvement in wall motion, in contrast to that reported by Schlack et al. [48]. CGS-21680 significantly reduced neutrophil accumulation in the area at risk, as well as inhibiting in vitro neutrophil superoxide radical production and neutrophil adherence to the endothelium of isolated coronary artery segments. These data provide an association between adenosine's anti-neutrophil effects and its infarct-sparing effect.

If the cardioprotective effects of adenosine specifically administered during reperfusion are related to its inhibitory actions on PMNs and endothelium, then the vascular compartment is a primary site of action of adenosine. Adenosine A₂ receptors are present and functional on both neutrophils and the vascular endothelium. To test the hypothesis that the vascular compartment is a primary site of adenosine actions against reperfusion injury, Todd et al. [49] used a large molecular weight adenosine congener (polyadenylic acid, PolyA) that contains only one adenosine moiety at its 3′ end, and is retained in the vascular compartment. A nearly sub-vasodilator dose of PolyA administered at reperfusion in a rabbit model of coronary occlusion-reperfusion reduced infarct size by 50%. Furthermore, the effects of PolyA were reversed by the adenosine receptor antagonist 8-SPT, confirming an adenosine receptor-mediated mechanism. However, infarct size was not altered by the highly A₁-selective antagonist 8-(3-noradamantyl)-1,3-dipropylxanthine (KW-3902, 1 mg/kg i.v.), implicating an A₂ receptor mechanism. In addition, PolyA significantly inhibited PMNs superoxide generation and adherence to coronary endothelium. This study [49] strongly suggested that the intravascular compartment is an important site for the cardioprotective actions of adenosine during reperfusion by inhibiting PMN-endothelial cell interactions.

Subsequent studies have largely corroborated the beneficial effects of adenosine in models of LAD occlusion followed by both short-term and long-term reperfusion. An adenosine analog, AMP579, which has both A₁ and A₂ receptor actions similar to that of native adenosine, but has a longer half-life, was administered at the onset of reperfusion and continued for 2 hours post-reperfusion [42]. AMP-579 reduced infarct size, attenuated the inflammatory response to ischemia-reperfusion mediated by neutrophil accumulation in parenchymal tissue and adherence to coronary artery endothelium, and preserved endothelial function. These actions of AMP-579 are entirely consistent with the primary effects of adenosine described from other studies.

Adenosine has been used as an adjunct to cardioplegia solutions. Partly because it reduces ischemic severity by opening K_{ATP} channels and hyperpolarizing the myocytes, and partly because of its potent anti-neutrophil effects. In 1976, Hearse et al. [50] reported that adenosine used as an adjunct to cardioplegia improved post-ischemic contractile function. Numerous studies have since investigated the efficacy of adenosine as an adjunct to crystalloid cardioplegia. Most of these studies showed significant improvement in post-ischemic contractile function compared to unsupplemented crystalloid counterparts. The beneficial effects of adenosine-enhanced crystalloid cardioplegia have been attributed to a number of mechanisms independent of neutrophil inhibition, including an augmentation in the rate of anaerobic glycolysis and energy status, a reduction in calcium accumulation resulting from cell hyperpolarization, and inhibition of endothelial cell activation.

The mechanistic action of adenosine as an adjunct to blood cardioplegia was first investigated by Hudspeth et al. [51, 52] in which adenosine was used as an adjunct to a standard hypothermic, hyperkalemic blood cardioplegic solution in ischemically injured hearts (30 minutes of normothermic global ischemia). Blood cardioplegia supplemented with 400 μM adenosine reversed the post-ischemic systolic dysfunction observed with unsupplemented blood cardioplegia. The protection was inhibited with the subtype non-specific adenosine antagonist 8-p-sulfophenyl theophylline (8-p-SPT), confirming a receptor-mediated mechanism. The potent anti-neutrophil effects of adenosine would suggest that significant cardioprotection would be exerted during reperfusion, and not necessarily during the period of cardioplegia itself. Hence, administration of the purine in hypothermic cardioplegia may not be the most effective environment.

Based on adenosine's potent inhibition of neutrophil-mediated reperfusion injury, Thourani et al. [53] tested the hypothesis that adenosine given during the period of reperfusion following aortic declamping would provide similar benefits to adenosine administered as an adjunct to blood cardioplegia. In a canine model of regional coronary occlusion, it was shown that adenosine administered either as an adjunct to blood cardioplegia (100 □M) alone or only during reperfusion (140 μg/kg/min) reduced infarct size, which is further described infra in connection with FIG. 4, improved post-ischemic contractile function, reduced myocardial edema, and attenuated neutrophil accumulation in the ischemia-reperfused area compared to the unsupplemented blood cardioplegia group. Furthermore, the hearts treated with adenosine only during reperfusion demonstrated better post-ischemic coronary artery endothelial function that was not observed with either unsupplemented blood cardioplegia or adenosine-enhanced blood cardioplegia. This observation is consistent with adenosine's potent anti-neutrophil effects.

Although the cooperative activation between platelets and neutrophils, leading to amplified neutrophil activation during ischemia and reperfusion, may be attenuated by adenosine [8, 37, 38], adenosine does not inhibit all processes associated with organ injury. A recent study showed that adenosine may indirectly inhibit thrombin-induced expression of tissue factor on endothelium [1, 2]; it has, however, little if any direct effect on protease-mediated activity, such as activation of vascular endothelium by the serine protease thrombin, and protease-stimulated cytokines.

Aprotinin in Cardioprotection

Unlike adenosine, aprotinin is a potent inhibitor of serine protease activity, including kallikrein, and thrombin. In a porcine closed-chest model of LAD occlusion and reperfusion, thrombin levels increased specifically during the reperfilsion phase. In addition to its effects on the coagulation cascade, thrombin is a direct activator of P-selectin expression on coronary vascular endothelial cells, which initiates the recruitment of neutrophils and other inflammatory cells in the pathogenesis of reperfusion injury [3]. Thrombin also stimulates platelets, which release cytokines that activate neutrophils, in addition to directly binding to neutrophils, thereby further supporting thrombin as a potential participant in the inflammatory response involving neutrophils. Studies support the hypothesis that thrombin may be a mediator of reperfusion injury through activation of coronary vascular endothelium, or by stimulating the generation of cytokines such as TNF□ [4]. Although aprotinin inhibits the extravasation of neutrophils, it does not inhibit early neutrophil adherence to coronary artery endothelium [12].

Aprotinin has been reported to reduce the physiological consequences of ischemia and reperfusion. Diaz et al. [56] reported in 1977 that aprotinin decreased myocardial infarction produced by a permanent (24 hours) coronary artery occlusion. Aprotinin was administered intravenously at 100,000 KIU 30 minutes after occlusion was imposed, i.e. during ischemia. Aprotinin decreased histologically apparent infarct size. In agreement, aprotinin treatment decreased creatine kinase activity in the area at risk myocardium, suggesting a reduction in morphologic injury, and consistent with the reduction in infarct size.

Transient coronary artery occlusion results in contractile dysfunction in the involved myocardium without necrosis. This “stunning” has been attributed to reversible abnormalities in sarcoplasmic reticular calcium transients and calcium regulation mechanisms. McCarthy et al. [57] tested the effects of aprotinin in a canine model of 15 minutes coronary artery occlusion, in which the aprotinin (30,000 KIU/kg bolus plus 7,000 KIU/kg/hr) was administered intravenously prior to occlusion, i.e. as a pretreatment before ischemia. There was a trend in the aprotinin group for the degree of systolic bulging to be less than in the control group, suggesting a reduced severity of ischemia. The aprotinin group showed significantly greater recovery of systolic function in the area at risk compared to the control group, which is further described infra in connection with FIG. 6. However, the study by McCarthy et al. [57] did not determine the mechanism by which pretreatment with aprotinin attenuated contractile dysfunction in this model of myocardial stunning. The study also did not determine the efficacy of aprotinin in attenuating reperfusion injury specifically since it was given before coronary occlusion. Similar results were reported by Hendrikx et al. [58] in an ovine model of myocardial stunning induced by 20 minutes of coronary occlusion and 1 hour of reperfusion.

Preliminary work by Pruefer et al. and Buerke et al. [59, 60] reported on the use of aprotinin in a rat model of coronary artery occlusion and 24 hours of reperfusion. In contrast to other studies, 5,000 or 20,000 KIU/kg aprotinin was administered before the onset of reperfusion, thereby targeting only the components of reperfusion injury, as opposed to ischemic injury as in the study of McCarthy et al. [57]. Infarct size, which is further described infra in connection with FIG. 7, estimated from creatine kinase loss from myocardium, was significantly reduced by both doses of aprotinin. In addition, the reduction in infarct size was associated with attenuation in neutrophil accumulation in the area at risk myocardium, and less extravascular infiltration at 24 hours, which is further described infra in connection with FIG. 8. Finally, aprotinin treatment attenuated the appearance of apoptosis in the area at risk myocardium. This study is significant in that it shows that aprotinin is effective against reperfusion injury events, which is entirely in keeping with a reduction in the inflammatory response during reperfusion.

A few studies have reported that aprotinin used in conjunction with a cardioplegic solution setting, particularly for long-term storage. In a study by Sunamori et al. [61], isolated canine hearts were administered multidose (q 1 hour) crystalloid cardioplegia containing 150 KIU aprotinin for 6 hours of arrest, followed by 2 hours of blood reperfusion from a donor system. There was no difference between the aprotinin-treated hearts and a control group in post-ischemic non-specific creatine kinase (CK) activity or CK-MB isoenzyme activity. Recovery of post-ischemic ATP after reperfusion was significantly greater in the aprotinin-treated group, with no differences in other high-energy phosphates. However, levels of the lysosomal enzyme N-acetyl-□-D-glucosaminidase in coronary sinus blood were significantly lower during reperfusion in the aprotinin group. In addition, morphologic damage was moderate in the control group, while it was largely minimal in the aprotinin group. Paradoxically, there was significantly less recovery of systolic function (end-systolic pressure-volume relationship) in the aprotinin-treated group. In summary, no clear picture of myocardial preservation was demonstrated in this study [61]. In contrast to the study of Sunamori et al. [61], Gurevitch et al. [62] showed significant protection with 105 KIU/L aprotinin administered as a pretreatment and adjunct to crystalloid cardioplegia. These conflicting data suggest that dose of aprotinin used is an important factor, and suggests the need for carefully conducted dose-response studies.

In a model of storage in cold cardioplegia, Bull et al. [63] reported that 200 KIU aprotinin attenuated the decline in ATP content and protein synthesis of rat myocardium slices incubated in cold (4° C.) crystalloid cardioplegia solution for up to 6 hours of storage. It was not clear from that study whether aprotinin maintained greater ATP concentration by improving myocardial synthesis of ATP, or by the reduction in the hydrolysis of ATP. Importantly, this study [63] reported that aprotinin suppressed both TNF□ generation and uptake by the myocardial tissue slices. These beneficial effects were achieved in a system free of inflammatory cells and plasma soluble elements, such as circulating thrombin, FVII/a, etc. The study of Gurevitch et al. [62] confirmed cardioprotection by high-dose aprotinin in a blood cell-free and plasma-free model of ischemia and reperfusion. There were several magnitudes of difference in the concentration of aprotinin used between the two studies. Again, the effective dose of aprotinin necessary to demonstrate cardioprotection during cardioplegia, either acutely or during prolonged cold storage, remains to be identified.

Aprotinin does not inhibit all processes associated with organ injury. It specifically inhibits protease-mediated injury and protease-stimulated responses, i.e. to thrombin, FV11a and FXa. Furthermore, the effective doses required to elicit cardioprotection are varied and may represent the varied etiology of mechanisms involved in organ ischemia-reperfusion injury.

Therefore, a heretofore unaddressed need exists in the art to address the aforementioned deficiencies and inadequacies.

### SUMMARY OF THE INVENTION

In one aspect, the present invention relates to a method of preventing organ ischemia-reperfusion injury. In one embodiment, the method includes administrating to a patient in need thereof a pharmaceutical composition comprising a serine protease inhibitor and adenosine, an adenosine agonist or a pharmaceutically acceptable derivative or prodrug or metabolite thereof.

The serine protease inhibitor is selected from the group consisting of 4-(2-aminoethyl)benzenesulfonylfluoride, □-amino-n-caproic acid, □₁-antichymotrypsin, antipain, antithrombin III, □₁-antitrypsin, p-amidinophenylmethylsulfonyl fluoride, aprotinin, cathepsin/subtilisin inhibitor (Boc-Val-Phe-NHO-Bz-pCl), chymostatin ([(S)-1-carboxy-2-phenylethyl]-carbamoyl-□-[2-amidohexahydro-4(S)-pyrimidyl]-(S)-glycyl-[A=Leu, B=Val, or C=Ile]-phenylalaninal), chymotrypsin inhibitor I, 3,4-dichloroisocoumarin, diisopropylfluorophosphate, dipeptidylpeptidase IV inhibitor I (Ile-Pro-Ile), dipeptidylpeptidase IV inhibitor II (H-Glu-(NHO-Bz)-Pyr), ecotin, elastase inhibitor I (Boc-Ala-Ala-Ala-NHO-Bz), □₂-macroglobulin, PPACK (D-Phe-Pro-Arg-chloromethylketone), PPACK II, N^{a}-tosyl-Lys chloromethyl ketone, N^{a}-tosyl-Phe chloromethyl ketone, and any mixture thereof. In a preferred embodiment, the serine protease inhibitor is aprotinin.

The adenosine agonist or pharmaceutically acceptable derivative is selected from the group consisting of AB-MECA (N⁶-4-amino benzyl-5′-N-methylcarboxamidoadenosine), CPA (N⁶-cyclopentyladenosine), ADAC (N⁶-[4-[[[4-[[[(2-aminoethyl)amino]carbonyl]methyl]-anilino]carbonyl]methyl]phenyl]adenosine), CCPA (2-chloro-N⁶-cyclopentyladenosine), CHA (N⁶-cyclohexyladenosine), GR79236 (N⁶-[1S,trans,2-hydroxycyclopentyl]adenosine), S-ENBA ((2S)—N⁶-(2-endonorbanyl)adenosine), IAB-MECA (N⁶-(4-amino-3-iodobenzyl)adenosine-5′-N-methylcarboxamidoadenosine), R—PIA (R—N⁶-(phenylisopropyl)adenosine), ATL146e (4-{3-[6-amino-9-(5-ethylcarbamoyl-3,4-dihydroxy-tetrahydro-furan-2-yl)-9H-purin-2-yl]-prop-2-ynyl}-cyclohexanecarboxylic acid methyl ester), CGS-21680 (APEC or 2-[p-(2-carbonyl-ethyl)-phenyl ethyl amino]-5′-N-ethylcarboxamidoadenosine), CV1808 (2-phenylaminoadenosine), HENECA (2-hex-1-ynyl-5′-N-ethylcarboxamido adenosine), NECA (5′-N-ethyl-carboxamido adenosine), PAPA-APEC (2-(4-[2-[(4-aminophenyl)methyl carbonyl]ethyl]phenyl)ethylamino-5′-N-ethyl carboxamidoadenosine), DITC-APEC (2-[p-(4-isothiocyanatophenylamino thiocarbonyl-2-ethyl)-phenylethylamino]-5′-N-ethylcarboxamidoadenosine), DPMA (N⁶-(2(3,5-dimethoxy phenyl)-2-(2-methyl phenyl)ethyl)adenosine), S—PHPNECA ((S)-2-phenylhydroxypropynyl-5′-N-ethylcarbox amidoadenosine), WRC-0470 (2-cyclohexylmethylidenehydrazinoadenosine), AMP-579 (1S-[1a,2b,3b,4a(S*)]]-4-[7-[[2-(3-chloro-2-thienyl)-1-methylpropyl]amino]-3H-imidazo[4,5-b]pyridyl-3-yl]cyclopentane carboxamide), IB-MECA (N⁶-(3-iodobenzyl)adenosine-5′-N-methyluronamide), 2-CIADO (2-chloroadenosine), I-ABA (N⁶-(4-amino-3-iodobenzyl)adenosine), S—PIA (S—N⁶-(phenylisopropyl)adenosine), 2-[(2-aminoethyl-aminocarbonylethyl)phenylethyl amino]-5′-N-ethyl-carboxamidoadenosine, 2-Cl—IB-MECA (2-chloro-N⁶-(3-iodobenzyl)adenosine-5′-N-methyluronamide), polyadenylic acid, and any mixture thereof.

In another aspect the present invention relates to a pharmaceutical composition. In one embodiment, the pharmaceutical composition includes a serine protease inhibitor and adenosine, an adenosine agonist or a pharmaceutically acceptable derivative or prodrug or metabolite thereof.

In yet another aspect, a method of preventing organ ischemia-reperfusion injury is provided that includes concomitantly administering to a patient in need thereof a serine protease inhibitor and adenosine, an adenosine agonist or a pharmaceutically acceptable derivative or prodrug or metabolite thereof.

In a further aspect, a method of preventing organ ischemia-reperfusion injury is provided that includes administering to a patient in need thereof sequentially in any order a serine protease inhibitor and adenosine, an adenosine agonist or a pharmaceutically acceptable derivative or prodrug or metabolite thereof.

In yet a further aspect, the present invention relates to a method of preventing organ or tissue injury at a predetermined point or period of intervention. In one embodiment, the method includes administrating to a patient in need thereof a pharmaceutical composition comprising a serine protease inhibitor and adenosine, an adenosine agonist or a pharmaceutically acceptable derivative or prodrug or metabolite thereof at the point on or about reperfusion, or before or during the ischemic or injury-inducing event.

The organ or tissue injury is related to at least one of cardiac surgery, non-surgical cardiac revascularization, organ transplantation, perfusion, ischemia, reperfusion, ischemia-reperfusion injury, oxidant injury, cytokine induced injury, shock induced injury, resuscitations injury and apoptosis. The shock induced injury can be hemorrhagic, septic, or traumatic injury, or any combination of them.

The administration is made at the predetermined point of time related to at least one of pre-treatment regimen, pharmacological preconditioning, and a reperfusion or post interventional therapy, wherein the pharmacological preconditioning is a treatment administered before the ischemic intervention followed by a brief period of reperfusion or washout before a lethal ischemia event.

In another aspect, the present invention relates to a method of preventing organ ischemia-reperfusion injury comprising administrating to a living subject in need thereof a pharmaceutical composition comprising a protease inhibitor and an agent that alters activities of G protein coupled receptors and cAMP, an analog or a pharmaceutically acceptable derivative or prodrug thereof.

In one embodiment of the present invention, the protease inhibitor is selected from the group consisting of 4-(2-aminoethyl)benzenesulfonylfluoride, □-ammo-n-caproic acid, □₁-antichymotrypsin, antipain, antithrombin III, □₁-antitrypsin, p-amidinophenylmethyl sulfonyl fluoride, aprotinin, cathepsin/subtilisin inhibitor (Boc-Val-Phe-NHO-Bz-pCl), chymostatin ([(S)-1-carboxy-2-phenylethyl]-carbamoyl-□-[2-amidohexahydro-4(S)-pyrimidyl]-(S)-glycyl-[A=Leu, B=Val, or C=Ile]-phenylalaninal), chymotrypsin inhibitor I, 3,4-dichloroisocoumarin, diisopropylfluoro phosphate, dipeptidylpeptidase IV inhibitor I (Ile-Pro-Ile), dipeptidylpeptidase IV inhibitor II (H-Glu-(NHO-Bz)-Pyr), ecotin, elastase inhibitor I (Boc-Ala-Ala-Ala-NHO-Bz), □₂-macroglobulin, PPACK (D-Phe-Pro-Arg-chloromethylketone), PPACK II, N^{a}-tosyl-Lys chloromethyl ketone, N^{a}-tosyl-Phe chloromethyl ketone, acetyl-pepstatin (Ac-Val-Val-(3S,4S)-Sta-Ala-(3S,4S)-Sta-OH), calpain inhibitor I (N-acetyl-Leu-Leu-norleucinal), calpain inhibitor II (N-acetyl-Leu-Leu-Met-CHO), amastatin ([(2S,2R)]-3-amino-2-hydroxy-5-methylhexanoyl]-Val-Val-Asp-OH), arphamenine A ((2R,5S)-5-amino-8-guanidino-4-oxo-2-phenylmethyloctanoic acid), arphamenine B ((2R,5S)-5-amino-8-guanidino-4-oxo-2-p-hydroxyphenylmethyloctanoic acid), benzamidine, bestatin ([(2S,2R)-3-amino-2-hydroxy-4-phenylbutanoyl]-L-Leucine), CA-074 ((L-3-trans-[propylcarbamoyl]oxirane-2-carbonyl)-L-isoleucyl-L-proline), CA-074-Me ((L-3-trans-[propylcarbamoyl]oxirane-2-carbonyl)-L-isoleucyl-L-proline-methylester), calpastatin, calpeptin (benzyloxycarbonylleucyl-norleucinal), carboxypeptidase inhibitor, cathepsin inhibitor I (Z-Phe-Gly-NHO-Bz), cathepsin inhibitor II (Z-Phe-Gly-NHO-Bz-pMe), cathepsin inhibitor III (Z-Phe-Gly-NHO-Bz-pOMe), cathepsin B inhibitor I (Z-Phe-Ala-CH₂F), cathepsin B inhibitor II (Ac-Leu-Val-lysinal), cathepsin L inhibitor I (Z-Phe-Phe-CH₂F), cathepsin L inhibitor II (Z-Phe-Tyr-CHO), cathepsin L inhibitor III (Z-Phe-Tyr-(t-Bu)-CHN₂), cathepsin L inhibitor IV (1-naphthalenesulfonyl-Ile-Trp-CHO), cathepsin L inhibitor V (Z-Phe-Tyr(OtBu)-COCHO), cathepsin L inhibitor VI (N-(4-biphenylacetyl)-S-methylcysteine-(D)-Arg-Phe-□-phenethylamide), cathepsin S inhibitor (Z-Phe-Leu-COCHO), cystatin, diprotin A (H-Ile-Pro-Ile-OH), E-64 (trans-epoxysuccinyl-L-leucylamido-(4-guanidino)butane), E-64 d (loxistatin, or (2S,3S)-trans-epoxysuccinyl-L-leucylamido-3-methylbutane ethyl ester), ebelactone A (3,11-dihydroxy-2,4,6,8,10,12-hexamethyl-9-oxo-6-tetradecenoic 1,3-lactone), ebelactone B (2-ethyl-3,11-dihydroxy-4,6,8,10,12-penta methyl-9-oxo-6-tetradecenoic 1,3-lactone), EDTA (ethylenediaminetetraacetic acid), EGTA (ethyleneglycol-bis(□-aminoethyl)-N,N,N′,N′-tetraacetic acid), elastase inhibitor II (MeOSuc-Ala-Ala-Pro-Ala-CMK), elastase inhibitor III (MeOSuc-Ala-Ala-Pro-Val-CMK), elastatinal (Leu-(Cap)-Gln-Ala-al or N—[(S)-1-carboxy-isopentyl)-carbamoyl-alpha-(2-iminohexahydro-4(S)-pyrimdyl]-L-glycyl-L-glutaminyl-L-alaninal), E-64 (loxistatin, or (2S,3S)-trans-epoxysuccinyl-L-leucylamido-3-methylbutane ethyl ester), N-ethyl maleimide, GGACK (1,5-dansyl-L-glutamyl-L-glycyl-L-arginine chloromethyl ketone), galardin (N-[(2S)-(methoxycarbonylmethyl)-4-methylpentanoyl]-L-tryptophan-methyl amide), 2-guanidinoethylmercaptosuccinic acid, hirudin, HIV protease inhibitor (Ac-Leu-Val-phenylalaninal), leuhistin (((2R,3S)-3-amino-2-hydroxy-2-(1H-imidazol-4-ylmethyl)-5-methyl)-5-methylhexanoic acid), leupeptin (acetyl-leucyl-leucyl-arginal), NCO-700, PEFABLOC SC (4-(2-aminoethyl)-benzenesulfonyl fluoride), pepstatin (isovaleryl-Val-Val-4-amino-3-hydroxy-6-methylheptanoyl-Ala-4-amino-3-hydroxy-6-methylheptanoic acid), phebestin ((2S,3R)-3-amino-2-hydroxy-4-phenylbutanoyl-L-valyl-L-phenylalanine), PMSF (phenyl methyl sulfonyl fluoride), phosphoramidon (N-alpha-L-rhamnopyranosyloxy(hydroxyl phosphinyl)-L-Leucyl-L-tryptophan, plummer's inhibitor (D,L-2-mercaptomethyl-3-guanidino-ethylthiopropanoic acid), 1,10-phenanthroline, subtilisin inhibitor I (Boc-Ala-Ala-NHO-Bz), subtilisin inhibitor II (Z-Gly-Phe-NHO-Bz), subtilisin inhibitor III (Z-Gly-Phe-NHO-Bz-pOMe), subtilisin inhibitor IV (Boc-Pro-Phe-NHO-Bz-pCl), subtilisin inhibitor V (Boc-Ala-Pro-Phe-NHO-Bz), TIMP-2 (tissue inhibitor of metalloproteinase 2), trypsin inhibitor, secretory leukocyte protease inhibitor, and any mixture thereof.

Moreover, the agent that alters activities of G protein coupled receptors and cAMP or pharmaceutically acceptable derivative is selected from the group consisting of AB-MECA (N⁶-4-amino benzyl-5′-N-methylcarboxamidoadenosine), CPA (N⁶-cyclopentyladenosine), ADAC (N⁶-[4-[[[4-[[[(2-aminoethyl)amino]carbonyl]methyl]-anilino]carbonyl]methyl]phenyl]adenosine), CCPA (2-chloro-N⁶-cyclopentyladenosine), CHA (N⁶-cyclohexyladenosine), GR79236 (N⁶-[1S,trans,2-hydroxycyclopentyl]adenosine), S-ENBA ((2S)—N⁶-(2-endonorbanyl)adenosine), IAB-MECA (N⁶-(4-amino-3-iodobenzyl)adenosine-5′-N-methylcarboxamidoadenosine), R—PIA (R—N⁶-(phenylisopropyl)adenosine), ATL146e (4-{3-[6-amino-9-(5-ethylcarbamoyl-3,4-dihydroxy-tetrahydro-furan-2-yl)-9H-purin-2-yl]-prop-2-ynyl}-cyclohexanecarboxylic acid methyl ester), CGS-21680 (APEC or 2-[p-(2-carbonyl-ethyl)-phenyl ethyl amino]-5′-N-ethylcarboxamidoadenosine), CV1808 (2-phenylaminoadenosine), HENECA (2-hex-1-ynyl-5′-N-ethylcarboxamido adenosine), NECA (5′-N-ethyl-carboxamido adenosine), PAPA-APEC (2-(4-[2-[(4-aminophenyl)methyl carbonyl]ethyl]phenyl)ethylamino-5′-N-ethyl carboxamidoadenosine), DITC-APEC (2-[p-(4-isothiocyanatophenylamino thiocarbonyl-2-ethyl)-phenylethylamino]-5′-N-ethylcarboxamidoadenosine), DPMA (N⁶-(2(3,5-dimethoxy phenyl)-2-(2-methyl phenyl)ethyl)adenosine), S—PHPNECA ((S)-2-phenylhydroxypropynyl-5′-N-ethylcarbox amidoadenosine), WRC-0470 (2-cyclohexylmethylidenehydrazinoadenosine), AMP-579 (1S-[1a,2b,3b,4a(S*)]]-4-[7-[[2-(3-chloro-2-thienyl)-1-methylpropyl]amino]-3H-imidazo[4,5-b]pyridyl-3-yl]cyclopentane carboxamide), IB-MECA (N⁶-(3-iodobenzyl)adenosine-5′-N-methyluronamide), 2-CIADO (2-chloroadenosine), I-ABA (N⁶-(4-amino-3-iodobenzyl)adenosine), S—PIA (S—N⁶-(phenylisopropyl)adenosine), 2-[(2-aminoethyl-aminocarbonylethyl)phenylethyl amino]-5′-N-ethyl-carboxamidoadenosine, 2-Cl—IB-MECA (2-chloro-N⁶-(3-iodobenzyl)adenosine-5′-N-methyluronamide), polyadenylic acid, adenosine, and any mixture thereof.

In yet another aspect, the present invention relates to a pharmaceutical composition that includes a protease inhibitor and an agent that alters activities of G protein coupled receptors and cAMP or a pharmaceutically acceptable derivative or prodrug or metabolite thereof.

In a further aspect, the present invention relates to a method of preventing organ ischemia-reperfusion injury that includes concomitantly administering to a living subject in need thereof a protease inhibitor and an agent that alters activities of G protein coupled receptors and cAMP or a pharmaceutically acceptable derivative or prodrug or metabolite thereof.

In yet a further aspect, the present invention relates to a method of preventing organ ischemia-reperfusion injury that includes administering to a living subject in need thereof sequentially in any order a protease inhibitor and an agent that alters activities of G protein coupled receptors and cAMP or a pharmaceutically acceptable derivative or prodrug or metabolite thereof.

The present invention in another aspect relates to a method of preventing organ or tissue injury at predetermined point or period of intervention comprising administrating to a living subject in need thereof a pharmaceutical composition comprising a protease inhibitor and an agent that alters activities of G protein coupled receptors and cAMP, an analog or a pharmaceutically acceptable derivative or prodrug or metabolite thereof.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

FIG. 1 depicts the level of tissue factor expression both qualitatively (Western blot analysis) and quantitatively (densitometry).

FIG. 2 depicts the interaction between neutrophils and endothelium involved in the inflammation process during reperfusion.

FIG. 3 depicts coronary artery endothelial function after co-incubation with neutrophils in the presence (dysfunction) or absence of thrombin.

FIG. 4 shows that infarct size (area of necrosis vs. area at risk ratio) is reduced most by adenosine given during the reperfusion phase (ADO-R) rather than as an additive to the cardioplegia solution (ADO-I) during experimental surgical revascularization for evolving infarction.

FIG. 5 shows adenosine added to the blood perfusing the ischemic myocardial vascular bed for the initial 30 minutes of reperfusion reduced the infarct size.

FIG. 6 shows that systolic function in the area at risk after reperfusion is significantly improved in the aprotinin-treated group.

FIG. 7 depicts infarct size, estimated from creatine kinase loss from myocardium, following aprotinin therapy.

FIG. 8 depicts neutrophil accumulation of ischemic-reperfused myocardium, estimated from the neutrophil-specific enzyme myeloperoxidase.

FIG. 9 is a schematic representation of one embodiment of the present invention depicting the process of systemic administration of aprotinin and intracoronary administration of adenosine.

FIG. 10 is a schematic representation of one embodiment of the present invention depicting the process of intracoronary administration of aprotinin and adenosine.

FIG. 11 is a schematic drawing of the chemical structures of some of the adenosine analogues disclosed for use in the present invention.

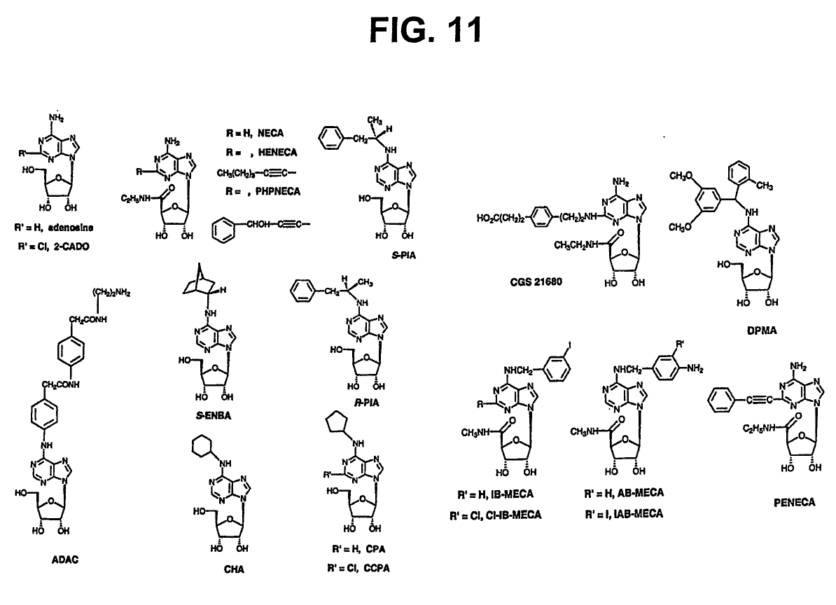

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. Referring to the drawings, like numbers indicate like components throughout the views. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention. Additionally, some terms used in this specification are more specifically defined below.

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to various embodiments given in this specification.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, the term “living subject” refers to a human being such as a patient, or an animal such as a lab testing monkey.

### Overview of the Invention

Among other things, applicants have invented the use of a serine protease inhibitor and adenosine when administered as a single pharmaceutical composition, concomitantly or sequentially in any order to a patient for the prevention of organ ischemia or reperfusion injury. The methods and compositions disclosed herein can be used in medical procedures including cardiac surgery, non-surgical cardiac revascularization, organ transplantation, perfusion, ischemia, reperfusion, ischemia-reperfusion, oxidant injury, cytokine induced injury, shock induced injury, resuscitation injury, or apoptosis.

Adenosine has a broad spectrum of physiological effects that make it suitable as a cardioprotective agent with effectiveness in all three therapeutic windows of opportunity (pretreatment, during ischemia, and reperfusion), and against numerous targets including the neutrophil and tissue factor. The duration of the physiological actions of adenosine seem to extend well beyond its plasma half-life. In addition, adenosine reduces reperfusion injury by inhibiting the neutrophil, the endothelium, and their interactions directly, largely by A₂ₐ-receptor mechanisms and transduction through the G-protein coupled system.

Aprotinin also inhibits a number of aspects of inflammation relevant to reperfusion injury. As non-limiting examples, aprotinin reduces superoxide anion production by activated neutrophils [64, 65]. This may be important as the generation of oxygen radicals, specifically of hydrogen peroxide, has been implicated in the pathogenesis of myocardial stunning. In addition, elastase activity, shown to be important in mediating myocyte injury during hypoxia-reperfusion [66], is inhibited by aprotinin. This action of aprotinin may attenuate the effects of this neutrophil-derived protease in ischemic myocardium. Furthermore, aprotinin attenuates extravasation of neutrophils across microvascular endothelium in response to chemokines such as IL-8, fMLP and platelet activating factor [12]. Aprotinin also inhibits the expression of endothelial cell adhesion molecules critical in the pathogenesis of reperfusion injury leading to necrosis, including ICAM-1, VCAM-1, but not E-selectin [67]. E-selectin has been implicated in the early adhesion responses between neutrophils and endothelium. Failure to attenuate this early adhesion molecule expression may explain the failure of aprotinin to attenuate neutrophil adhesion [12]. This would be one reason to partner adenosine with aprotinin, since adenosine attenuates expression of early phase adhesion molecules like P-selectin and E-selectin. Aprotinin inhibits the surface expression of β₂-integrins CD11a/CD18, CD11b/CD18 and CD11c/CD18 on neutrophils [68] while at the same time it also inhibits the shedding of L-selectin [69] which is critical to the rolling of neutrophils along the endothelial surface, and key to the transendothelial migration of neutrophils. Another serine protease inhibitor, secretory leukocyte protease inhibitor (SLPI), has been shown to inhibit nuclear translocation of NF-κB in a pulmonary immune response. Since NF-κB is a key transcription activation factor in the inflammatory responses involving cytokines and chemokines, this may be another way in which aprotinin exerts a cardioprotective mechanism. Aprotinin has also been shown to inhibit complement activation [70], inhibit the generation and release of TNFα [63, 71, 72], decrease agonist-induced expression of GP11b-IIIa receptors, and affect the expression of P-selectin, depending on the stimulus and environment.

Recent reports by Gabazza et al. [1, 2] suggest that adenosine has a direct inhibitory effect on tissue factor expression on endothelial cells. Adenosine also inhibits the amplification of tissue factor expression induced by thrombin itself. Hence, in combination with its inhibitory effects on P-selectin expression initiated by thrombin, adenosine may directly attenuate the generation of thrombin and the thrombin-initiated inflammatory cascade, as well as other effects listed above. Therefore, the present invention provides complementary actions on the inflammatory response initiated during ischemia-reperfusion and cardiopulmonary bypass, thereby conferring broader cardioprotective actions and/or allows lower concentrations of each individual drug to be used to achieve the same or similar results.

Clinical Applications

The methods and compositions of the present invention can be used as (a) a pretreatment regimen, (b) a form of pharmacological preconditioning, in which the treatment is administered before the ischemic or injury inducing intervention followed by a brief period of reperfusion (washout), and/or (c) a reperfusion or post-interventional therapy. The treatment can be used in cardiac surgery (on-pump or off-pump), in non-surgical revascularization in the cardiac cath-lab setting using catheter-based therapy, in transplantation, or to other organs undergoing transplantation, perfusion or reperfusion, or other treatment. Examples of organ perfusion includes, but is not limited to, selective perfusion of the kidneys during abdominal aortic repair, aortic perfusion of visceral organs during deep hypothermic circulatory arrest, retrograde or antegrade perfusion to the brain during deep hypothermic circulatory arrest or surgical-based or catheter-based vascular intervention of cerebral vessels. The treatment can also be applied to whole body ischemia and reperfusion caused by hemorrhage, shock and resuscitation.

Perfusion of the target vessel immediately after anastomosis would avoid ischemia and allow the delivery of drugs selectively to the target segment to avoid reperfusion injury, vasodilate the vasculature and avoid arrhythmias. Recently, Guyton et al. [54] reported a method for perfusing the target vessel after the distal anastomosis was complete, but before the proximal anastomosis was constructed. The vascular graft was connected to a microprocessor-controlled, servo-regulated, constant flow pump system such as Myocardial Protection System by Quest Medical, Inc. Allen, Tex. that allows control of flow rate while monitoring perfusion pressure. With this technique, coined Perfusion-Assisted Direct Coronary Artery Bypass (PADCAB), ischemia could be truncated and drugs could be added to the blood perfusate. Hemodynamic stability was improved by servo-perfusion, especially when hypotension accompanied cardiac positional manipulation.

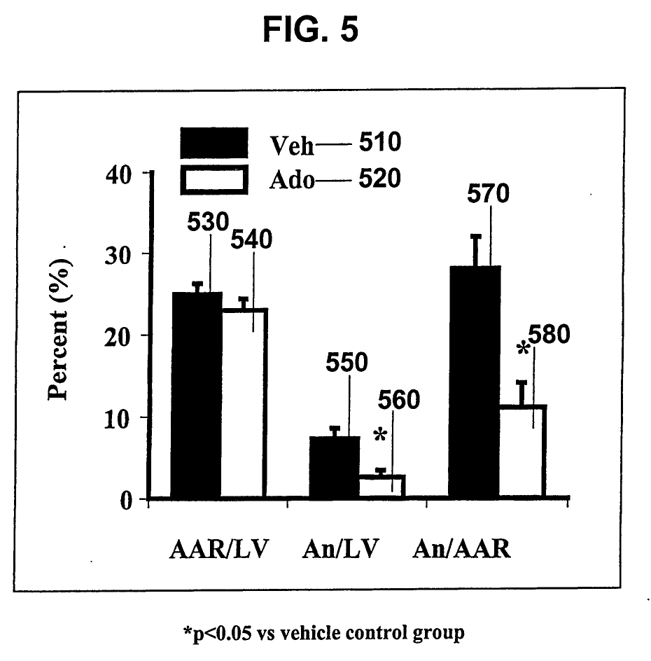

Muraki et al. [55) demonstrated that this same servo-perfusion technique can be used to introduce intracoronary cardioprotective agents to the revascularized segment to avoid reperfusion injury. Using a model of severe coronary occlusion that causes contractile dysfunction, infarction and edema in the area at risk, as well as severe endothelial dysfunction in the target vessel, adenosine (10 μM) was added to the blood perfusing the ischemic vascular bed for the initial 30 minutes of reperfusion. After two hours of reperfusion, this very brief treatment with intracoronary adenosine reduced infarct size, which is further described infra in connection with FIG. 5, attenuated neutrophil accumulation and edema in the area at risk, and avoided endothelial dysfunction in the ischemic-reperfused LAD compared to a group reperfused in similar manner but without adjunctive adenosine. Because of the selective nature of delivery of this otherwise potent vasodilator, there was no hypotension associated with intracoronary delivery of adenosine.

Routes of Administration

In the cath-lab setting, the methods and compositions can be administered intravenously or by catheter-based techniques, or a combination thereof, with or without associated delivery devices (i.e. pumps). In cardiac surgery, the treatment can be administered intravenously, in or associated with cardioplegia solutions, via local delivery procedures including direct injection into grafts or native arteries, and via perfusion-assisted techniques (i.e. perfusion-assisted direct coronary artery bypass, PADCAB, technology). The compositions of the present invention can be infused intravenously, while other agents are delivered to the target organ selectively, or both can be delivered by either intravenous or intravascular selective administration.

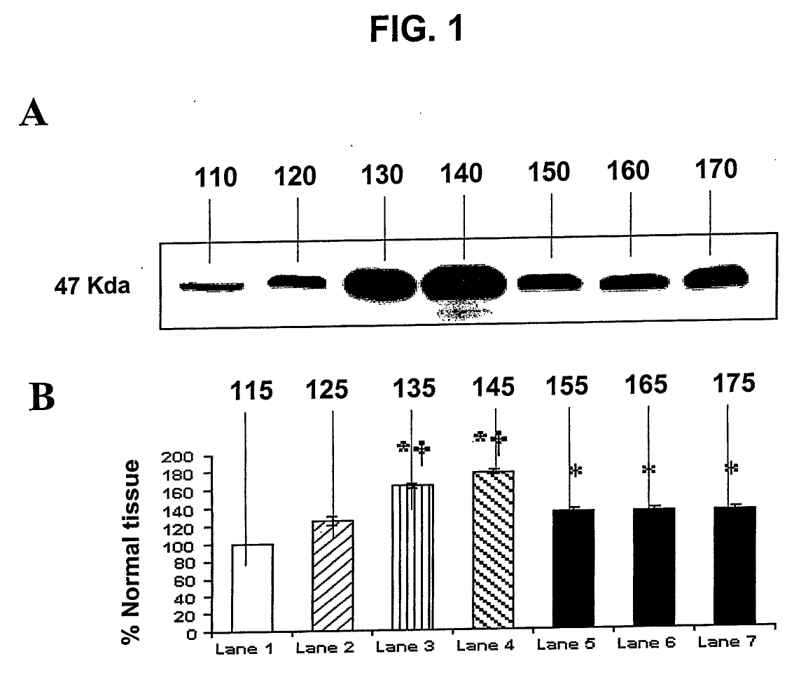

Referring now to FIG. 1, a configuration demonstrating the expression levels of tissue factor (TF) in different tissues both qualitatively and quantitatively, according to an embodiment of the invention, is illustrated. The qualitative expression level of TF as visualized by Western blot analysis is shown in FIG. 1(A). The corresponding quantitation expression level of TF as visualized by densitometry is shown in FIG. 1(B). Normal myocardium was used as a control to show the baseline of TF expression in normal tissue. In FIG. 1(A), it is represented by the thinnest band 110 and corresponds to the lowest bar in FIG. 1(B) with a percentage reading of 100% normal tissue 115. The non-ischemic left ventricular myocardium contralateral to the area at risk showed slightly higher expression of TF, demonstrated by a slightly thicker band 120 and slightly taller bar 125 than those of the normal myocardium 110 and 115, respectively. Non-necrotic area at risk after 75 minutes LAD occlusion and reperfusion and necrotic area at risk after 75 minutes LAD occlusion and reperfusion showed significant increase of TF expression level, as demonstrated by the thickest bands 130 and 140 and corresponding highest bars 135 and 145, respectively. Myocardium after 75 minutes LAD occlusion in the absence of reperfusion showed no significant elevation in expression of TF compared to the normal myocardium and the non-ischemic left ventricular myocardium contralateral to the area at risk, as demonstrated by only slightly thicker bands 150, 160, and 170 and no significant elevation in corresponding bars 155, 165, and 175, respectively. After 75 minutes LAD occlusion, myocardial ischemia followed by reperfusion 130/135 and 140/145 showed markedly elevated TF expression level compared to myocardium in the absence of reperfusion 150/155, 160/165, and 170/175, suggested that TF expression was initiated primarily after the onset of reperfusion.

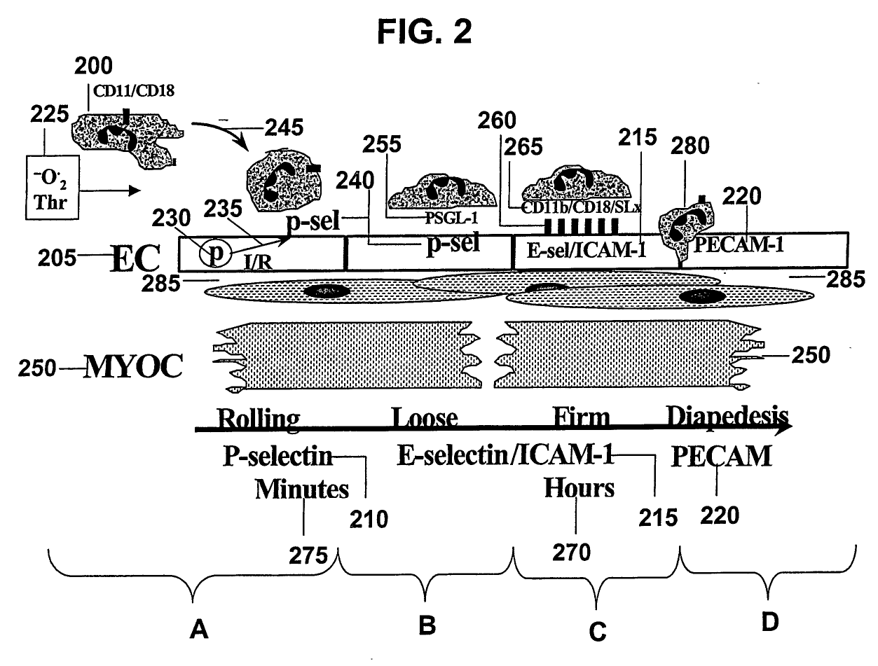

Referring now to FIG. 2, a configuration of the interactions between polymorphonuclear neutrophils (PMNs) 200 and coronary vascular endothelium (EC) 205 involved in the inflammation process during reperfusion, according to an embodiment of the invention, is illustrated. The interactions begin immediately upon reperfusion, and may continue for a period of time or over 72 hours. The interactions are mediated by a highly specific and temporally orchestrated sequence of events involving the early P-selectin 210, 230 and 240) and late (ICAM-1 215, VCAM (not shown), PECAM 220) expression of adhesion molecules on both the endothelium and PMNs. According to the time course of the inflammation during reperfusion, these events can be classified into four continuous phases or stages A, B, C and D as shown in FIG. 2, respectively. During the early moments of reperfusion and/or inflammation, in a rolling phase A, in response to oxygen radical species, the serine protease thrombin (Thr), C5a, TNFα, and IL-1 [21-23] 225, P-selectin that is stored as preformed granules 230 in the Weibel-Palade bodies (not shown) is rapidly translocated 235 to the endothelial surface and expressed on the lumenal surface as P-sel 240. The pro-adhesive properties of the vascular endothelium are also stimulated [16-19]. Interaction with P-selectin 240 on endothelium causes the neutrophil to start rolling 245 [17, 24] towards the endothelium. This “rolling phenomenon” plays a critical role in the pathogenesis of the early phase of reperfusion injury in the myocardium 250 [25]. In a loose attachment phase B, the rolling neutrophil starts to attach loosely 255 on the endothelial surface. After the initial loose tethering of PMNs to the vascular endothelium, the process enters a firm attachment phase C. Firm adherence is facilitated by interaction 260 between CD11b/CD18 265 on PMNs and ICAM-1 215 on the endothelium. ICAM-1 is constitutively expressed at low levels, but de novo protein synthesis and surface expression is stimulated by cytokines (i.e. TNFα) beginning at 4-6 hours after reperfusion 270, and peaking at 24 hours. This later response is in contrast to the early (<30 minutes) expression of P-selectin 275. In a diapedesis phase D, transendothelial migration of PMNs 280 into the interstitium 285 such as smooth muscle cells provides direct access to cardiomyocytes 250. The applicant's previous studies and pilot data confirm that endothelial ICAM-1 expression in myocytes occurs later than 24-72 hours [15, 34]. The early P-selectin-dependent PMNs adherence to endothelium is prerequisite to a constellation of pathophysiological processes that ultimately lead to infarction, contractile dysfunction, microvascular injury, endothelial cell dysfunction, and apoptosis. However, the continued interaction between neutrophils and endothelium in later phases of reperfusion (6-72 hours) leads to expansion of necrosis and no-reflow zones, and the initiation of apoptosis [35]. The development of apoptosis has been reported by us and others to be triggered primarily during reperfusion, and is therefore a “reperfusion event” [36].

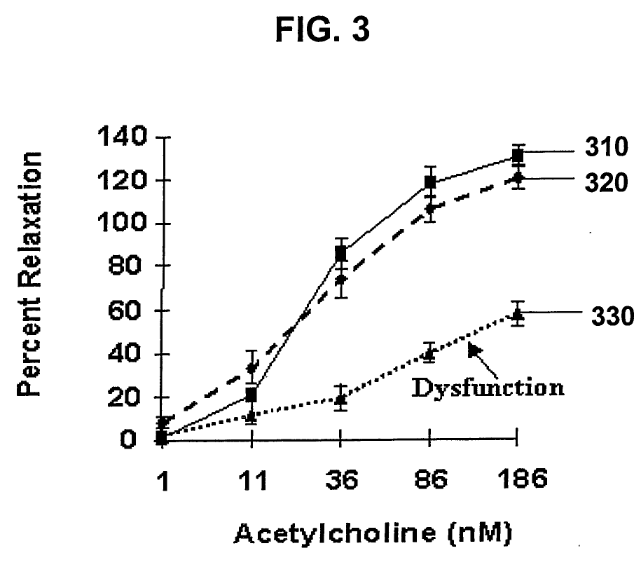

Referring now to FIG. 3, coronary artery endothelial function after co-incubation with neutrophils in the presence or absence of thrombin, according to an embodiment of the invention, is illustrated. The percent of coronary artery relaxation at different acetylcholine concentrations were used to indicate the state of coronary artery endothelium function. In a control where normal segments of coronary artery were not treated with either neutrophils or thrombin, the percent of coronary artery relaxation increases significantly with increased acetylcholine concentration as indicated by solid line 310. Co-incubation of neutrophils with coronary artery segments that have not been activated with thrombin resulted in similar degree of increase as shown by dashed line 320 indicating no dysfunction. Co-incubation of neutrophils with coronary artery segments that have been activated with thrombin, however, resulted in significantly less endothelium-derived relaxation indicated by dotted line 330. The significantly less percentage of coronary artery relaxation in response to increased acetylcholine concentration indicated endothelial dysfunction [8, 21, 24, 26-33].

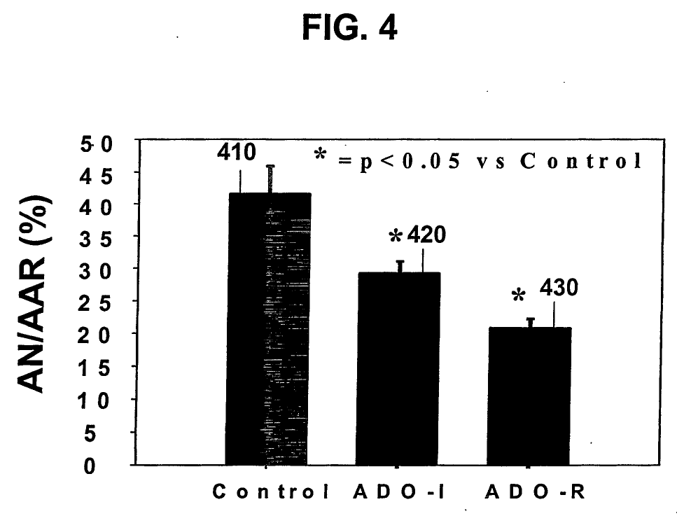

Referring now to FIG. 4, the effect of adenosine administeredas an adjunct to blood cardioplegia (ADO-I) or when given only at reperfusion (ADO-R), according to an embodiment of the invention, is illustrated. Relative infarct size (AN/AAR %) is measured by the area of necrosis (AN) vs. area at risk (AAR) ratio in percentage. In a model of regional coronary occlusion followed by surgically imposed reperfusion, control relative infarct size was measured where adenosine was not used in any phase of the operation as indicated by histogram bar 410. When adenosine was administered as an adjunct to blood cardioplegia (100 μM) alone (ADO-I) during elective arrest, less relative infarct size was observed in post-ischemic myocardium as shown by histogram bar 420. The smallest relative infarct size was observed when adenosine was administered (140 μg/kg/min) during reperfusion only (ADO-R) as indicated by histogram bar 430.

Referring now to FIG. 5, results of adenosine added to the blood perfusing the ischemic vascular bed for the initial 30 minutes of reperfusion reduced the infarct size, according to an embodiment of the invention, are given. Using a canine model of severe coronary occlusion that causes contractile dysfunction, infarction and edema in the area at risk, as well as severe endothelial dysfunction in the target vessel, vehicle control group without adenosine (Veh, indicated with solid bars 510) and a group with adenosine (Ado, indicated with open bars 520) (10 μM) added to the blood perfusing the ischemic vascular bed in the first 30 minutes of reperfusion, were compared. All data are illustrated with relative percentages. In both groups, the area at risk (AAR) size as percent of the left ventricular mass (LV) was very similar (AAR/LV) as indicated by similar height of histogram bars 530 for vehicle and 540 for adenosine treated group. After two hours of reperfusion, infarct sizes (An) as a percent of LV (An/LV) or AAR (An/ARR) were measured and compared. The percentages of infarct sizes from adenosine treated hearts have significantly lower An/LV (bar 560) than the vehicle (bar 550). Similarly, the percentages of infarct sizes from adenosine treated hearts had significantly lower An/ARR (bar 580) than the vehicle (bar 570). Because of the selective nature of delivery of this otherwise potent vasodilator, there was no hypotension associated with intracoronary delivery of adenosine.

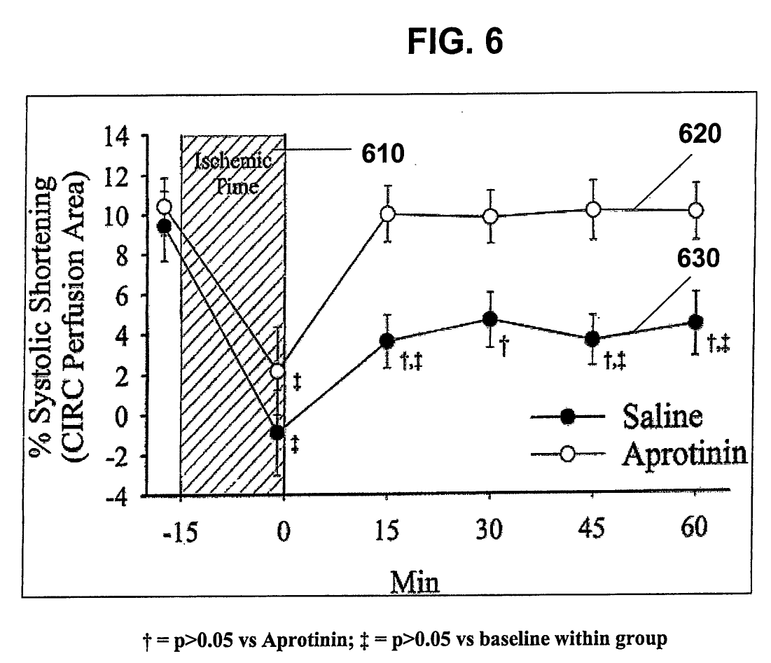

As shown in FIG. 6, a systolic function in the area at risk after reperfusion is significantly improved in the aprotinin-treated group. McCarthy et al. [57] tested the effects of aprotinin in a canine model of 15 minutes coronary artery occlusion 610, in which the aprotinin (30,000 KIU/kg bolus plus 7,000 KIU/kg/hr) was administered intravenously prior to occlusion, i.e. as a pretreatment before ischemia. Saline was administered similarly in a control group. The aprotinin group, indicated by open circle 620 showed significantly greater recovery of systolic function in the area at risk (CIRC perfusion area) compared to the control group, indicated by solid circle 630 as demonstrated by the higher percentage of systolic shortening.

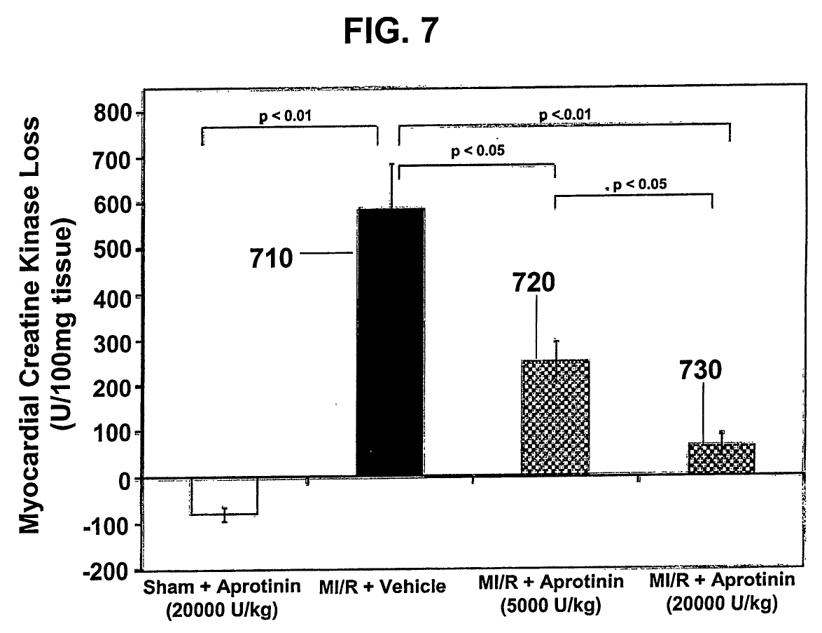

Referring now to FIG. 7, results of infarct size estimated from creatine kinase loss from myocardium following aprotinin therapy, according to an embodiment of the invention are given. Aprotinin was used in a rat model of coronary artery occlusion and 24 hours of reperfusion. 5,000 KIU/kg or 20,000 KIU/kg aprotinin was administered before the onset of reperfusion, thereby targeting only the components of reperfusion injury. Vehicle without aprotinin showed the most pronounced myocardial creatine kinase loss as indicated by the tallest histogram bar 710. Infarct size in the 5,000 KIU/kg aprotinin treatment group was significantly reduced as shown by the lower level of myocardial creatine kinase loss as indicated by a lower bar 720 compare to the control group 710. Infarct size estimated from myocardial creatine kinase loss was further significantly reduced at 20,000 KIU/kg dose of aprotinin as indicated by the lowest bar 730, lower than control 710 and lower than the data obtained from 5,000 KUI/kg dosage 720. Aprotinin therapy therefore, reduced creatine kinase activity and infarct size reduction in a dose-dependent manner.

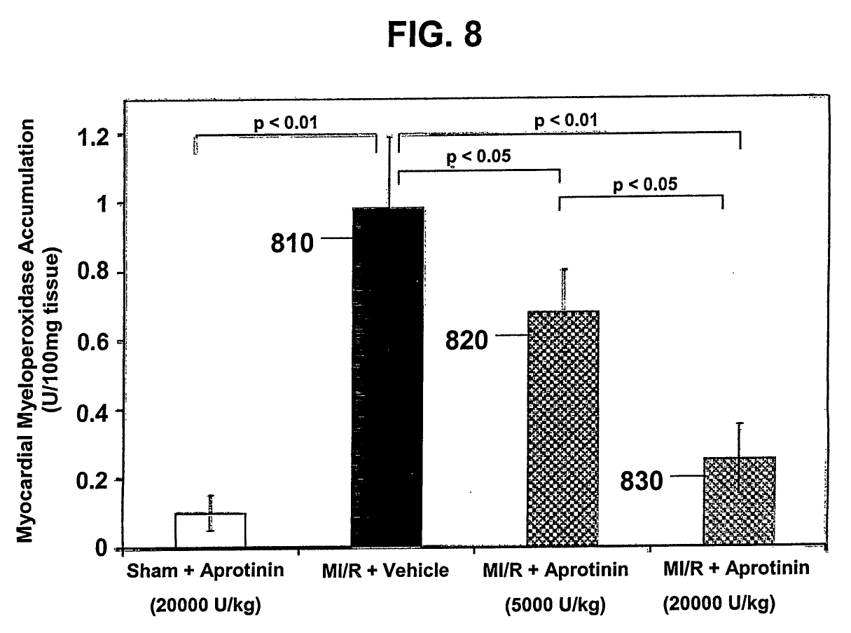

FIG. 8 provides neutrophil accumulation in ischemic-reperfused myocardium, estimated from the neutrophil-specific enzyme myeloperoxidase, according to an embodiment of the invention. Aprotinin was used in the same rat model of coronary artery occlusion and 24 hours of reperfusion described for FIG. 7. 5,000 KIU/kg or 20,000 KIU/kg aprotinin was administered before the onset of reperfusion, thereby targeting only the components of reperfusion injury. Vehicle without aprotinin showed the most pronounced neutrophil-specific myocardial myeloperoxidase accumulation as indicated by the tallest histogram bar 810. Infarct size at 5,000 KIU/kg aprotinin treatment was significantly reduced as shown by the lower level of myocardial myeloperoxidase accumulation as indicated by a bar 820 that is lower than the control bar 810. Infarct size estimated from myocardial myeloperoxidase accumulation was significantly reduced at 20,000 KIU/kg dose of aprotinin as indicated by the shortest bar 830 that is shorter than the control bar 810 as well as the 5,000 KIU/kg dosage bar 820. Aprotinin therapy, therefore, reduced myocardial myeloperoxidase neutrophil accumulation and infarct size in a dose-dependent manner.

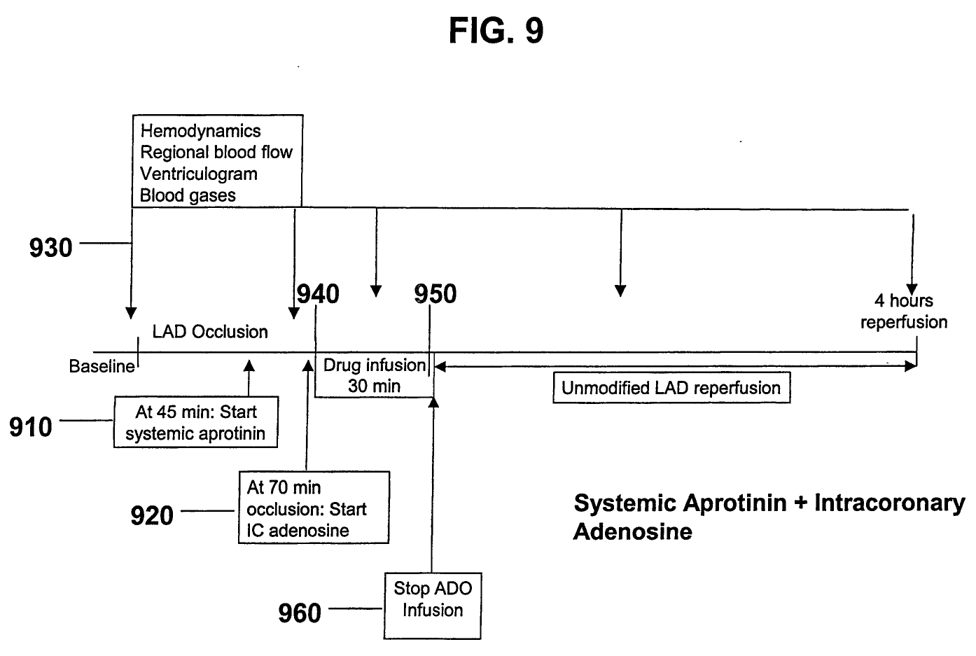

Referring now to FIG. 9, a process of systemic administration of aprotinin and intracoronary administration of adenosine, according to an embodiment of the present invention, is illustrated. Intravenous (systemic administration) aprotinin is envisioned at this stage because it is associated with few complications, in contrast to adenosine, which has numerous complications and loss of efficacy when administered intravenously in this situation (i.e. off-pump or cath-lab). The aprotinin will be loaded by intravenous slow bolus (one-half hour duration) about 45 minutes (time point 910) after the start of ischemia (time point 930), and discontinued at the start of reperfusion (time point 940). In determining the efficacy of combined aprotinin-adenosine therapy, intracoronary adenosine treatment will be given at about 70 minutes (time point 920), i.e. 5 minutes before the start of reperfusion (time point 940) because of the clinical relevance of this timing. The adenosine infusion will continue for about 30 minutes (time point 950), i.e. will stop at 100 minutes (time point 960) after the start of ischemia (time point 930).

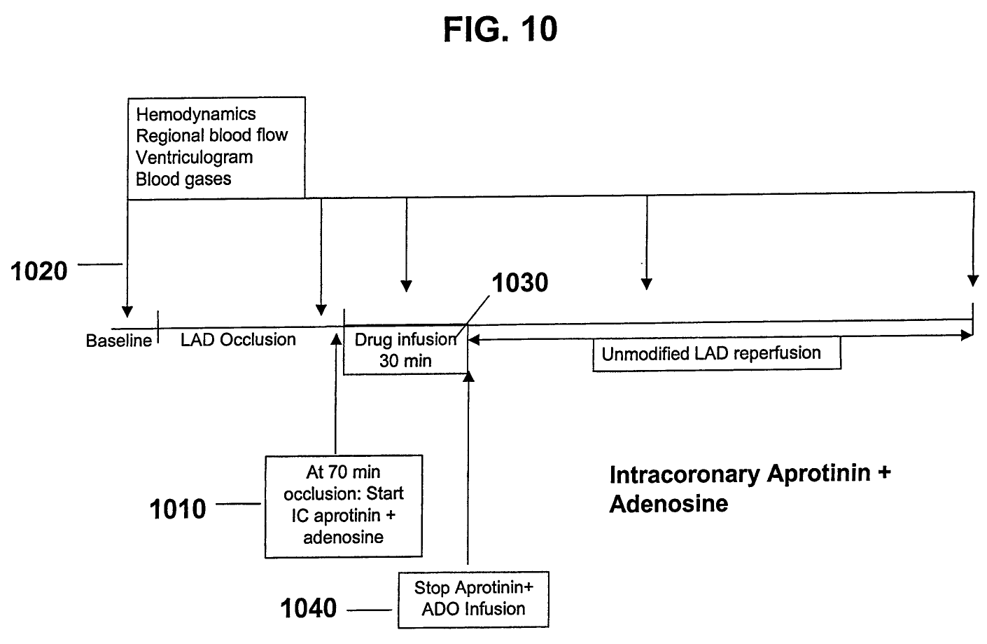

Referring now to FIG. 10, a process of intracoronary administration of aprotinin and adenosine simultaneously, according to an embodiment of the invention, is illustrated. Intracoronary administration of aprotinin and adenosine will start simultaneously at 70 minutes (time point 1010) after the start of ischemia (time point 1020). The infusion will continue for 30 minutes (time point 1030), i.e. will stop at 100 minutes (time point 1040) after the start of ischemia (time point 1020) or 25 minutes into reperfusion.

### Methods and Implementations

Without intent to limit the scope of the invention, additional exemplary methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as data are processed, sampled, converted, or the like according to the invention without regard for any particular theory or scheme of action.

### EXAMPLES

### Example 1

### Non-Surgical Ischemia-Reperfusion Injury in a Closed-Chest Porcine Model

The use of combined adenosine and aprotinin treatment in the cath-lab setting was performed in a closed-chest porcine model of regional ischemia and reperfusion. Farm-bred pigs were initially anesthetized with ketamine, xylazine, acepromazine, diazepam and atropine, followed by maintenance anesthesia with inhaled isoflurane. Through a small femoral artery cut-down, a pigtail catheter was fluoroscopically guided into the left ventricle for injection of non-radioactive microspheres to measure regional myocardial blood flow. A similar cut-down was performed on the contralateral femoral artery, through which was placed a sheath to introduce a 7-Fr guide catheter and angioplasty-type balloon catheters. The 7-Fr guide catheter was inserted into this sheath and fluoroscopically guided to the left main coronary artery. The left main coronary ostium was engaged by the catheter and a guide wire. An angioplasty-type balloon catheter was guided into the LAD just distal to the first diagonal branch. Placement of the balloon was verified by intracoronary contrast dye injection, and documented by film capture. Intravenous amiodarone (8-10 mg/kg) was administered to control arrhythmias due to the coronary occlusion or subsequent reperfusion.

After all instrumentation was complete, the animal was allowed to stabilize for 10 minutes. Baseline hemodynamics (arterial pressure, heart rate) and myocardial blood flow (microspheres) were measured. The microspheres were injected via the pigtail catheter to quantify myocardial blood flow during steady state. Simultaneously a reference sample was withdrawn from the contralateral femoral artery through the side port of the sheath. The arterial reference sample was used to calculate blood flow by setting up a ratio of microspheres in a reference sample withdrawn at a known rate (by calibrated pump) to microspheres obtained in the tissue area of interest. The angioplasty balloon was inflated to totally occlude the LAD coronary artery distal to the first diagonal branch, and occlusion was maintained for 75 minutes, targeting an infarct size of approximately 40% of the area at risk. When ventricular fibrillation occurred, DC counter shocks were delivered by external paddles to convert the heart to normal sinus rhythm. After 75 minutes of balloon inflation, either vehicle (saline), adenosine alone, aprotinin alone or combined adenosine-aprotinin was delivered through the central lumen of the angioplasty catheter using an infusion pump set to deliver the drug(s) for the initial 30 minutes of reperfusion. Preliminary experiments in other models have shown that 30 minutes is effective in reducing reperfusion injury in the myocardial area at risk [55].

### Example 2

### Adenosine in the Prevention of Non-Surgical Ischemia-Reperfusion Injury

Non-surgical ischemia-reperfusion injury induced in a closed chest porcine model as described in Example 1 was carried out. Delivery of adenosine during the first 30 minutes of reperfusion was confirmed by microspheres infused at about 15 minutes of reperfusion, i.e. at the mid-point of adenosine-aprotinin infusion. Experiments have been conducted in controls (n=12), ischemic preconditioning (n=2), and intracoronary adenosine treatment at reperfusion (n=4, in which adenosine was infused at approximately 10-20 μM LAD blood concentration for about 30 minutes). In controls, there was no intervention at the time of reperfusion. The ischemic preconditioning protocol was conducted to determine whether infarct size in this closed chest model could be decreased by a well-known and well-characterized treatment, before unknown treatments were tested. In this paradigm, the 75 minutes of LAD occlusion was preceded by 2 cycles each consisted of about 5 minutes LAD occlusion followed by about 10 minutes of reperfusion.

From all experiments, the rate of fibrillation has been 6%, with 60% being converted and 40% being intractable. In controls (n=12), the area at risk has averaged 29.8±3.7% of the left ventricular mass; infarct size has averaged 41±6 of the area at risk. In the adenosine treated hearts, delivery of intracoronary adenosine during the first 30 minutes of reperfusion was verified by vasodilation in the area at risk (neutron microspheres) at about 15 minutes of (re)perfusion. In three of the four experiments, the delivery of adenosine via the intracoronary catheter was questionable since there was no vasodilatory effect during the 15 minutes reperfusion measurement. Infarct size in this group of three animals was not different from controls (40.4±3.6%). However, in the experiment in which delivery of adenosine was confirmed by vasodilation at 15 minutes reperfusion, the infarct size was 10%.

### Example 3

### Examination of Alternative Timing of Treatment: Reperfusions and Pretreatment

The basic porcine closed-chest model described in Example 1 will be used in the following studies. In these preliminary studies determining the efficacy of combined aprotinin-adenosine therapy, treatment will be given at the start of reperfusion because of the clinical relevance of this timing. Additional studies can then be performed to determine optimal timing, therapy at reperfusion only vs. pretreatment (pre-ischemic) therapy. Intravenous aprotinin can be used at this stage because it is associated with few systemic complications, in contrast to adenosine that has numerous complications and loss of efficacy when administered intravenously in this situation (i.e. off-pump). Other studies can examine intracoronary aprotinin as an alternative to intravenous aprotinin.

### Example 4

### Effective Dose of Aprotinin that Reduces Reperfusion Injury (Infarct Size)

Non-surgical ischemia-reperfusion injury induced in a closed chest porcine model as described in Example 1 will be carried out. The study groups are: control (n=8, no treatment will be initiated before reperfusion) and i.v. aprotinin of at least 2 groups with 2 different doses (e.g. n=8, 30,000 KIU/kg; n=8, 10,000 KIU/kg). The aprotinin is loaded by intravenous slow bolus (one-half hour duration) about 45 minutes after the start of ischemia, and discontinuing about 30 minutes later at the start of reperfusion. Infarct size will be measured to determine the most effective dose of aprotinin that reduces reperfusion injury.

### Example 5

### Combination: of Intracoronary Adenosine Plus Intravenous Aprotinin

Non-surgical ischemia-reperfusion injury induced in a closed chest porcine model as described in Example 1 will be carried out. The effective intracoronary dose of adenosine has been estimated from previous studies. The following experiments will confirm the efficacy of the combination of intracoronary adenosine with intravenous aprotinin. One group will receive intracoronary adenosine. 10-2,000 μM adenosine will be administered intracoronary beginning about 5 minutes prior to reperfusion, i.e. about 70 minutes after occlusion. Another group will receive systematic administered aprotinin about 45 minutes after the start of ischemia. The concentration of aprotinin will range from 200-1,000 KIU/mL of blood, calculated based on approximate LAD blood flow during the first 30 minutes of reperfusion. The third treatment group will receive a combination i.v. aprotinin+intracoronary adenosine. The concentrations of adenosine and aprotinin will be determined empirically based upon preliminary tests in separate groups of animals. As described supra in connection to FIG. 9, the aprotinin is loaded by intravenous slow bolus (one-half hour duration) about 45 minutes 910 after the start of ischemia 930, and discontinuing about 30 minutes later at the start of reperfusion. Intracoronary adenosine treatment will be given at about 70 minutes 920, 5 minutes before the start of reperfusion 940 because of the clinical relevance of this timing. The adenosine infusion will continue for about 30 minutes 950, i.e. will stop at 100 minutes 960 after the start of ischemia 930. The duration of infusion of adenosine will vary depending on optimal reduction of infarct size.

### Example 6

### Combination of Intracoronary Adenosine and Aprotinin

Non-surgical ischemia-reperfusion injury induced in a closed chest porcine model as described in Example 1 will be carried out. The effective intracoronary dose of adenosine has been estimated from previous studies. The following experiments will confirm the efficacy of the combination of intracoronary adenosine with aprotinin. One group will receive intracoronary adenosine. 10-2,000 μM adenosine will be administered intracoronary beginning about 5 minutes prior to reperfusion. Another group will receive aprotinin administered intracoronary beginning 5 minutes prior to reperfusion. The most efficacious dose from Example 4 will be used in this study. The third treatment group will receive a combination intracoronary adenosine and intracoronary aprotinin. The concentrations of adenosine and aprotinin will be determined empirically based upon preliminary tests in separate groups of animals. As described supra in connection to FIG. 10, intracoronary administration of aprotinin adenosine will start simultaneously at about 70 minutes 1010 after the start of ischemia 1020. The infusion will continue for about 30 minutes 1030, i.e. will stop at 100 minutes 1040 after the start of ischemia 1020.

### Example 7

### End Point Determinations for All Studies

The following endpoints will be used to determine the efficacy of the treatments described in the above examples. Infarcts size will be determined by TTC vital staining. Plasma creatine kinase activity is used to confirm TTC staining data and to determine the time course of tissue injury. The extent of tissue edema is also measured. Microvascular blood flow by microspheres (5 time points: baseline, end of ischemia, 15, 120 and 240 minutes reperfusion) is utilized to determine whether the extent of microvascular injury and no-reflow has been attenuated in the area at risk with treatments. It will also determine the amount of collateral blood flow in the area at risk during ischemia, which may influence infarct size. Tissue myeloperoxidase activity can be used as a marker of neutrophil accumulation in the area at risk vs. non-ischemic myocardium. This will establish an anti-inflammatory mechanism of individual treatments as well as combined treatment, which may show synergistic anti-neutrophil effects. Histological determination of location of neutrophils, i.e. intravascular vs. interstitial location will be used to comment on transmigration of neutrophils. Regional function of the anterior myocardium will be analyzed by regional analysis of contrast ventriculogram. The degree of apoptosis in the area at risk myocardium vs. non-ischemic left ventricle myocardium will be quantified, as well as the mechanistic marker proteins Bc1-2, Bax and caspases to determine mechanism of potential reduction of apoptosis. Thromboelastogram (TEG) measurements will be performed at baseline, and after each hour of reperfusion. In addition, platelet aggregation studies will be performed for concentration-response relationships of aprotinin, adenosine and the combination therapy, using collagen, ADP, EPI and thrombin as platelet activators.

### Example 8

### In Vitro Studies

The following studies will establish the effects of individual and combined aprotinin and adenosine effects on inflammatory cell processes shown to be important in the pathogenesis of ischemia-reperfusion injury. All studies will be performed using a dose-response approach to determine effective concentrations of each component vs. combined components.

a. Neutrophil superoxide generation in response to platelet activating factor;

b. Neutrophil adherence to coronary vascular endothelium;

c. Chemotaxis of neutrophils through endothelial-lined membrane. (Studies from Ken Taylor's group [12] have shown that aprotinin does not inhibit adherence to endothelium, but does attenuate chemotaxis to some extent. The process of chemotaxis is partially dependent on adherence. Therefore, inhibition of adherence by more completely inhibiting chemotaxis may be an advantage of combined therapy. These studies may demonstrate that the combination of adenosine and aprotinin overcomes the inability of aprotinin alone to inhibit endothelial adherence that leads to transmigration. These data will be associated with neutrophil accumulation data observed in the in vivo studies above.);

d. Endothelial activation state (The level of P-selectin and E-selectin expression will be determined by immunohistochemical staining since adenosine has been shown to attenuate endothelial cell activation [73], as has aprotinin [67]. It would be worthwhile to determine if the combination of the two more effectively reduces endothelial cell activation.);

e. Interaction between neutrophils and platelets. (Neutrophils are activated during reperfusion. Platelets release a number of factors that activate neutrophils, so the interactions between the two cell types may exacerbate the inflammatory component of ischemia-reperfusion injury. Neutrophils are co-incubated with platelets, and the degree of neutrophil activation is determined along with superoxide anion generation, adherence to endothelium, and resultant endothelial damage.).

While there has been shown several and alternate embodiments of the present invention, it is to be understood that certain changes can be made as would be known to one skilled in the art without departing from the underlying scope of the invention as is discussed and set forth in the specification given above and in the claims given below. Furthermore, the embodiments described above are only intended to illustrate the principles of the present invention and are not intended to limit the scope of the invention to the disclosed elements. Additionally, the references listed herein are incorporated into the application by reference for providing background information only.

### LIST OF REFERENCES

[1] Deguchi H, Takeya H, Urano H, Gabazza E C, Zhou H, Suzuki K. Adenosine regulates tissue factor expression on endothelial cells. Thromb Res 1998; 91(2):57-64.
[2] Gabazza E C, Hayashi T, Ido M, Adachi Y, Suzuki K. Adenosine inhibits thrombin-induced expression of tissue factor on endothelial cells by a nitric oxide-mediated mechanism. Clin Sci (Colch) 2002; 102:167-175.
[3] Kaur J, Woodman R C, Ostrovsky L, Kubes P. Selective recruitment of neutrophils and lymphocytes by thrombin: a role for NF-kB. Am J Physiol (Heart Circ Physiol) 2001; 281:H784-H795.
[4] Cirino G, Cicala C, Bucci M R, Sorrentino L, Maraganore J M, Stone S R. Thrombin functions as an inflammatory mediator through activation of its receptor. The Journal of Experimental Medicine 1996; 183(3):821-827.
[5] Fernandez A Z, Williams M W, Jordan J E, Zhao Z-Q, Vinten-Johansen J. Neutrophil (PMN) adherence to thrombin stimulated coronary vascular endothelium is inhibited by an adenosine (ADO) A₂-receptor mechanism. FASEB Journal 10, A611. 1996.
[6] Jordan J E, Zhao Z-Q, Sato H, Taft S, and Vinten-Johansen J. Adenosine A₂ receptor activation attenuates reperfusion injury by inhibiting neutrophil accumulation, superoxide generation and coronary endothelial adherence. J Pharmacol Exp Ther 1997; 280(1):301-309.
[7] Zhao Z-Q, Sato H, Vinten-Johansen J. Adenosine A₂-receptor activation inhibits neutrophil-mediated injury to coronary endothelium by attenuating superoxide anion generation and adherence. Circulation (Suppl) 90 (No. 4, Part 2), I-372. 1994.
[8] Zhao Z-Q, Sato H, Williams M W, Fernandez A Z, Vinten-Johansen J. Adenosine A₂-receptor activation inhibits neutrophil-mediated injury to coronary endothelium. Am J Physiol 1996; 271(4 Pt. 2):H1456-H1464.
[9] Zhao Z-Q, Nakamura M, Wang N-P, Wilcox J N, Shearer S T, Guyton R A Vinten-Johansen J. Administration of adenosine during reperfusion reduces injury of vascular endothelium and death of myocytes. Coron Artery Dis. 1999; 10(8):617-28.
[10] Zhao Z-Q, Todd J C, Sato H, Ma X-L, Vinten-Johansen J. Adenosine inhibition of neutrophil damage during reperfusion does not involve K(ATP)-channel activation. Am J Physiol 1997; 273(4 Pt. 2):H1677-H1687.
[11] Zhao Z-Q, Nakamura M, Wang N-P, Wilcox J N, Shearer S T, Katzmark S et al. Adenosine during early reperfusion reduces neutrophil-mediated necrosis, apoptosis and vascular dysfunction. FASEB Journal 13[4], A518. 1999.
[12] Asimakopoulos G, Thompson R, Nourshargh S, Lidington E A, Mason J C, Ratnatunga C P et al. An anti-inflammatory property of aprotinin detected at the level of leukocyte extravasation. Journal Thoracic Cardiovascular Surg 2000; 120:361-369.
[13] Jennings R B, Reimer K A. Acute myocardial ischemia: effects of reperfusion with arterial blood. Artif Cells Blood Substit Immobil Biotechnol 1994; 22:253-278.
[14] Reimer K A, Jennings R B. The “wavefront phenomenon” of myocardial ischemic cell death. II. Transmural progression of necrosis within the framework of ischemic bed size (myocardial at risk) and collateral flow. Lab Invest 1979; 40:633-644.
[15] Zhao Z-Q, Nakamura M, Wang N-P, Wilcox J N, Katzmark S, Guyton R A et al. Infarct extension and dynamic coronary endothelial dysfunction in the late reperfusion phase. Circulation 1998; 98(17): 1-796.
[16] Poullis M, Manning R, Laffan M, Haskard D O, Taylor K M, Landis R C. The antithrombotic effect of aprotinin: actions mediated via the protease-activated receptor 1. The Journal of Thoracic and Cardiovascular Surgery 2000; 120(2):370-378.
[17] Kubes P. Polymorphonuclear leukocyte-endothelium interactions: a role for pro-inflammatory and anti-inflammatory molecules. [Review]. Can J Physiol Pharmacol 1993; 71:88-97.
[18] Argenbright L W. Inflammatory mediators, cytokines, and leukocyte adhesion in postcapillary venules. In: Granger D N, Schmid-Schönbein G W, editors. Physiology and Pathophysiology of Leukocyte Adhesion. New York: Oxford University Press, 1995: 171-184.
[19] Deisher T A, Garcia I, Harlan J M. Cytokine-induced adhesion molecule expression on human umbilical vein endothelial cells is not regulated by cyclic adenosine monophosphate accumulation. Life Sci 1993; 53:365-370.
[20] Erlich J H, Boyle E M, Labriola J, Kovacich J C, Santucci R A, Fearns C et al. Inhibition of the tissue factor-thrombin pathway limits infarct size after myocardial ischemia-reperfusion injury by reducing inflammation. Am J Pathol 2000; 157(6):1849-1862.
[21] Davenpeck K L, Gauthier T W, Albertine K H, Lefer A M. Role of P-selectin in microvascular leukocyte-endothelial interaction in splanchnic ischemia-reperfusion. Am J Physiol 1994; 267:H622-30.
[22] Foreman K E, Vaporciyan A A, Bonish B K, Jones M L, Johnson K J, Glovsky M M et al. C5a-induced expression of P-selectin in endothelial cells. J Clin Invest 1994; 94(3):1147-1155.
[23] Lefer A M. Role of selectins in myocardial ischemia-reperfusion injury. [Review]. Ann Thorac Surg 1995; 60:773-777.
[24] Sheridan F M, Cole P G, Ramage D. Leukocyte adhesion to the coronary microvasculature during ischemia and reperfusion in an in vivo canine model. Circulation 1996; 93:1784-1787.
[25] Lefer A M, Tsao P S, Lefer D J, Ma X-L. Role of endothelial dysfunction in the pathogenesis of reperfusion injury after myocardial ischemia. FASEB J 1991; 5:2029-2034.
[26] Jordan J E, Zhao Z-Q, Vinten-Johansen J. The role of neutrophils in myocardial ischemia-reperfusion injury. Cardiovas Res 1999; 43:860-878.
[27] Lefer A M, Ma X-L, Weyrich A, Lefer D J. Endothelial dysfunction and neutrophil adherence as critical events in the development of reperfusion injury. Agents Actions Suppl 1993; 41:127-135.
[28] Lefer D J, Nakanishi K, Vinten-Johansen J, Ma X-L, Lefer A M. Cardiac venous endothelial dysfunction after myocardial ischemia and reperfusion in dogs. Am J Physiol 1992; 263:H850-H856.
[29] Boyle E M, Pohlman T H, Cornejo C J, Verrier E D. Endothelial cell injury in cardovascular surgery: Ischemia-reperfusion. Ann Thorac Surg 1996; 62:1868-1875.
[30] Tsao P S, Aoki N, Lefer D J, Johnson G, III, Lefer A M. Time course of endothelial dysfunction and myocardial injury during myocardial ischemia and reperfusion in the cat. Circulation 1990; 82:1402-1412.
[31] Geng J-G, Bevilacqua M P, Moore K L, McIntyre T M, Prescott S M, Kim J M et al. Rapid neutrophil adhesion to activated endothelium mediated by GMP-140. Nat 1990; 343:757-760.
[32] Jerome S N, Dore M, Paulson J C, Smith C W, Korthuis R J. P-selectin and ICAM-1-dependent adherence reactions: role in the genesis of postischemic no-reflow. Am J Physiol 1994; 266:H1316-21.
[33] Kubes P, Jutila M, Payne D. Therapeutic potential of inhibiting leukocyte rolling in ischemia/reperfusion. J Clin Invest 1995; 95:2510-2519.
[34] Golino P, Ragni M, Cirillo P, Scognamiglio A, Ravera A, Buono C, Guarino A, Piro O, Lambiase C, Botticella F, Ezban M, Condorelli M, Chiariello M. Recombinant human, active site-blocked factor VIIa reduces infarct size and the no-reflow phenomenon in rabbits. Am J Physiol Heart Circ Physiol. 2000; 278(5):H1507-16.
[35] Zhao Z-Q, Nakamura M, Wang N—P, Velez D A, Hewan-Lowe K O, Guyton R A et al. Dynamic progression of contractile and endothelial dysfunction and infarct extension in the late phase of reperfusion. J Surg Res 2000; 94:1-12.
[36] Zhao Z-Q, Nakamura M, Wang N-P, Wilcox J N, Shearer S, Ronson R S et al. Reperfusion induces myocardial apoptotic cell death. Cardiovasc Res 2000; 45:651-660.
[37] Cronstein B N, Kramer S B, Weissmann G, Hirschhorn R. Adenosine: a physiological modulator of superoxide anion generation by human neutrophils. J Exp Med 1983; 158:1160-1177.
[38] Cronstein B N, Rosenstein E D, Kramer S B, Weissmann G, Hirschhorn R. Adenosine: a physiologic modulator of superoxide anion generation by human neutrophils. Adenosine acts via an A₂ receptor on human neutrophils. J Immunol 1985; 135(12):1366-1371.
[39] Jordan J E, Thourani V H, Auchampach J A, Robinson J A, Wang N-P, Vinten-Johansen J. A₃ adenosine receptor activation attenuates neutrophil function and neutrophil-mediated reperfusion injury. Am J Physiol 1999; 277:H1895-H1905.
[40] Olafsson B, Forman M B, Puett D W, Pou A, Cates C U, Friesinger G C et al. Reduction of reperfusion injury in the canine preparation by intracoronary adenosine: importance of the endothelium and the no-reflow phenomenon. Circulation 1987; 76(5):1135-1145.
[41] Toombs C F, McGee D S, Johnston W E, Vinten-Johansen J. Myocardial protective effects of adenosine. Infarct size reduction with pretreatment and continued receptor stimulation during ischemia. Circulation 1992; 86:986-994.
[42] Budde J M, Velez D A, Zhao Z-Q, Clark K L, Morris C D, Muraki S et al. Comparative study of AMP579 and adenosine in inhibition of neutrophil-mediated vascular and myocardial injury during 24 hours of reperfusion. Cardiovas Res 2000; 47:294-305.
[43] Nakamura M, Zhao Z-Q, Clark K L, Guyton R A, Vinten-Johansen J. AMP579, a new adenosine analog, inhibits neutrophil O₂-generation, degranulation, adherence, and neutrophil-induced injury to coronary vascular endothelium by A_{2A} receptor mechanism. Circulation 100[18], I-832. 1999.
[44] Nakamura M, Zhao Z-Q, Clark K L, Velez D V, Guyton R A, Vinten-Johansen J. A novel adenosine analog, AMP579, inhibits neutrophil activation, adherence and neutrophil-mediated injury to coronary vascular endothelium. Eur J Pharmacol. 2000; 397(1): 197-205.
[45] Zhao Z-Q, Clark K L, Wang N-P, Velez D A, Guyton R A, Vinten-Johansen J. Comparison of AMP579 and adenosine in inhibition of cell-cell interaction between human neutrophil and vascular endothelial cell. Drug Devel Res 2000; 49:266-272.
[46] Babbitt D G, Virmani R, Forman M B. Intracoronary adenosine administered after reperfusion limits vascular injury after prolonged ischemia in the canine model. Circulation 1989; 80:1388-1399.
[47] Babbitt D G, Virmani R, Vildibill H D, Jr., Norton E D, Forman M B. Intracoronary adenosine administration during reperfusion following 3 hours of ischemia: effects on infarct size, ventricular function, and regional myocardial blood flow. Am Heart J 1990; 120:808-818.
[48] Schlack W, Schäfer M, Uebing A, Schäfer S, Borchard U, Thämer V. Adenosine A₂-receptor activation at reperfusion reduces infarct size and improves myocardial wall function in dog heart. J Cardiovasc Pharmacol 1993; 22:89-96.
[49] Todd J C, Zhao Z-Q, Williams M W, Sato H, Van Wylen D G L, Vinten-Johansen J. Intravascular adenosine at reperfusion reduces infarct size and neutrophil adherence. Ann Thorac Surg 1996; 62:1364-1372.
[50] Hearse D J, Stewart D A, Braimbridge M V. Cellular protection during myocardial ischemia. The development and characterization of a procedure for the induction of reversible ischemic arrest. Circulation 1976; 54:193-202.
[51] Hudspeth D A, Nakanishi K, Vinten-Johansen J. Zhao Z-Q, McGee D S, Hammon J W, Jr. Enhancement of blood cardioplegia with adenosine avoids postischemic dysfunction in severely injured hearts. Surg Forum 1993; 44:252-254.
[52] Hudspeth D A, Nakanishi K, Vinten-Johansen J, Zhao Z-Q, McGee D S, Williams M W et al. Adenosine in blood cardioplegia prevents postischemic dysfunction in ischemically injured hearts. Ann Thorac Surg 1994; 58:1637-1644.
[53] Thourani V H, Ronson R S, Van Wylen D G L, Shearer S T, Katzmark S L, Zhao Z-Q et al. Adenosine-supplemented blood cardioplegia attenuates postischemic dysfunction after severe regional ischemia. Circulation 1999; 100 [suppl II]:II-376-II-383.
[54] Guyton R A, Thourani V H, Puskas J D, Shanewise J S, Steele M A, Palmer-Steele C L et al. Perfusion-assisted direct coronary artery bypass: selective graft perfusion in off-pump cases. Ann Thorac Surg 2000; 69:171-175.
[55] Muraki S, Morris C D, Budde J M, Velez D A, Zhao Z-Q, Guyton R A et al. Experimental off-pump coronary artery revascularization with adenosine enhanced reperfusion. J Thorac Cardiovasc Surg 2001; 121(3):570-579.
[56] Diaz P E, Fishbein M C, Davis M A, Askenazi J, Maroko P R. Effect of the kallikrein inhibitor aprotinin on myocardial ischemic injury after coronary occlusion in the dog. Am J Cardiol 1977; 40(4):541-549.
[57] McCarthy R J, Tuman K J, O'Connor C, Ivankovich A D. Aprotinin pretreatment diminishes postischemic myocardial contractile dysfunction in dogs. Anesth Analg 1999; 89(5):1096.
[58] Hendrikx M, Rega F, Jamaer L, Valkenborgh T, Gutermann H, Mees U. Na⁺/H⁺-exchange inhibition and aprotinin administration: promising tools for myocardial protection during minimally invasive CABG. Europ J Cardio Thorac Surg 2001; 19:633-639.
[59] Buerke M, Pruefer D, Dahm M, Oelert H, Darius H, Meyer J. Serine protease inhibitor (Aprotinin) attenuates myocardial necrosis and apoptosis following ischemia and long-term reperfusion in rats. European Heart Journal 20[P996], 163. 1999.
[60] Pruefer D, Makowski J, Buerke M, et al. Aprotinin (Trasylol) attenuates reperfusion injury. FASEB Journal 15[462-8], A 569. 2001.
[61] Sunamori M, Sultan I, Suzuki A. Effect of aprotinin to improve myocardial viability in myocardial preservation followed by reperfusion. Ann Thorac Surg 1991; 52:971-978.
[62] Gurevitch J, Barak J, Hochhauser E, Paz Y, Yakirevich V. Aprotinin improves myocardial recovery after ischemia and reperfusion. Effects of the drug on isolated rat hearts. J Thorac Cardiovasc Surg 1994; 108:109-118.
[63] Bull D A, Connors R C, Albanil A, Reid B B, Neumayer L A, Nelson R et al. Aprotinin preserves myocardial biochemical function during cold storage through suppression of tumor necrosis factor. J Thorac Cardiovasc Surg 2000; 119:242-250.
[64] Lord R A, Roath O S, Thompson J F, Chant A D B, Francis J L. Effect of aprotinin on neutrophil function after major vascular surgery. Br J Surg 1992; 79:517-521.
[65] Hallett M B, Shandall A, Young H L. Mechanism of protection against “reperfusion injury” by aprotinin. Roles of polymorphonuclear leucocytes and oxygen radicals. Biochem Pharma 1995; 34(10):1757-1761.
[66] Buerke M, Weyrich A S, Lefer A M. Isolated cardiac myocytes are sensitized by hypoxia-reoxygenation to neutrophil-released mediators. Am J Physiol 1994; 266:H128-H136.
[67] Asimakopoulos G, Lidington E A, Mason J C, Haskard D O, Taylor K M, Landis R C. Effect of aprotinin on endothelial cell activation. Journal Thoracic Cardiovascular Surg 2001; 122(1):123-128.
[68] Asimakopoulos G, Kohn A, Stefanou D C, Haskard D O, Landis R C, Taylor K M. Leukocyte integrin expression in patients undergoing cardiopulmonary bypass. The Society of Thoracic Surgeons 2000; 69(4): 1192-1197.
[69] Asimakopoulos G, Taylor K M, Haskard D O, Landis R C. Inhibition of neutrophil L-selectin shedding: a potential anti-inflammatory effect of aprotinin. Perfusion 2000; 15(6):495-499.
[70] Himmelfarb J, Holbrook D, McMonagle E. Effects of Aprotinin on complement and granulocyte activation during ex vivo hemodialysis. Am J of Kidney Diseases 1994; 24(6):901-906.
[71] Hill G E, Alonso A, Spurzem J R, Stammers A H, Robbins R A. Aprotinin and methylprednisolone equally blunt cardiopulmonary bypass-induced inflammation in humans. J Thorac Cardiovasc Surg 1995; 110:1658-1662.
[72] Englberger L, Kipfer B, Berdat P A, Nydegger U E, Carrel T P. Aprotinin in coronary operation with cardiopulmonary bypass: does “low-dose” aprotinin inhibit the inflammatory response? Ann Thorac Surg 2002; 73(6):1897-1904.
[73] Zhao Z-Q, Sato H, Vinten-Johansen J. Adenosine inhibits neutrophil adherence and neutrophil-mediated damage to coronary endothelium. FASEB Journal 8, A634. 1994.

1. A method of preventing organ ischemia or reperfusion injury comprising administrating to a living subject in need thereof a pharmaceutical composition comprising:
a. a serine protease inhibitor; and
b. adenosine, an adenosine agonist or a pharmaceutically acceptable derivative or prodrug or metabolite thereof.

2. The method of claim 1, wherein the serine protease inhibitor is selected from the group consisting of 4-(2-aminoethyl)benzenesulfonylfluoride, ε-amino-n-caproic acid, α₁-antichymotrypsin, antipain, antithrombin III, α₁-antitrypsin, p-amidinophenylmethyl sulfonyl fluoride, aprotinin, cathepsin/subtilisin inhibitor (Boc-Val-Phe-NHO-Bz-pCl), chymostatin ([(S)-1-carboxy-2-phenylethyl]-carbamoyl-α-[2-amidohexahydro-4(S)-pyrimidyl]-(S)-glycyl-[A=Leu, B=Val, or C=Ile]-phenylalaninal), chymotrypsin inhibitor I, 3,4-dichloroisocoumarin, diisopropylfluoro phosphate, dipeptidylpeptidase IV inhibitor I (Ile-Pro-Ile), dipeptidylpeptidase IV inhibitor II (H-Glu-(NHO-Bz)-Pyr), ecotin, elastase inhibitor I (Boc-Ala-Ala-Ala-NHO-Bz), macroglobulin, PPACK (D-Phe-Pro-Arg-chloromethylketone), PPACK II, N^{a}-tosyl-Lys chloromethyl ketone, N^{a}-tosyl-Phe chloromethyl ketone, and any mixture thereof.

3. The method of claim 1, wherein the adenosine agonist or pharmaceutically acceptable derivative is selected from the group consisting of AB-MECA (N⁶-4-aminobenzyl-5′-N-methyl carboxamidoadenosine), CPA (N⁶-cyclopentyladenosine), ADAC (N⁶-[4-[[[4-[[[(2-aminoethyl)amino]carbonyl]methyl]-anilino]carbonyl]methyl]phenyl]adenosine), CCPA (2-chloro-N⁶-cyclopentyladenosine), CHA (N⁶-cyclohexyladenosine), GR79236 (N⁶-[1S,trans,2-hydroxy cyclopentyl]adenosine), S-ENBA ((2S)—N⁶-(2-endonorbanyl)adenosine), IAB-MECA (N⁶-(4-amino-3-iodobenzyl)adenosine-5′-N-methylcarboxamidoadenosine), R—PIA (R—N⁶-(phenyl isopropyl)adenosine), ATL146e (4-{3-[6-amino-9-(5-ethylcarbamoyl-3,4-dihydroxy-tetrahydro-furan-2-yl)-9H-purin-2-yl]-prop-2-ynyl}-cyclohexanecarboxylic acid methyl ester), CGS-21680 (APEC or 2-[p-(2-carbonyl-ethyl)-phenyl ethyl amino]-5′-N-ethylcarboxamidoadenosine), CV1808 (2-phenylaminoadenosine), HENECA (2-hex-1-ynyl-5′-N-ethylcarboxamido adenosine), NECA (5′-N-ethyl-carboxamido adenosine), PAPA-APEC (2-(4-[2-[(4-aminophenyl)methyl carbonyl]ethyl]phenyl)ethylamino-5′-N-ethyl carboxamidoadenosine), DITC-APEC (2-[p-(4-isothiocyanatophenylamino thiocarbonyl-2-ethyl)-phenylethylamino]-5′-N-ethylcarboxamido adenosine), DPMA (N⁶-(2(3,5-dimethoxy phenyl)-2-(2-methyl phenyl)ethyl)adenosine), S—PHPNECA ((S)-2-phenylhydroxypropynyl-5′-N-ethylcarbox amidoadenosine), WRC-0470 (2-cyclohexylmethylidenehydrazinoadenosine), AMP-579 (1S-[1a,2b,3b,4a(S*)]]-4-[7-[[2-(3-chloro-2-thienyl)-1-methylpropyl]amino]-3H-imidazo[4,5-b]pyridyl-3-yl]cyclopentane carboxamide), IB-MECA (N⁶-(3-iodobenzyl)adenosine-5′-N-methyluronamide), 2-CIADO (2-chloroadenosine), I-ABA (N⁶-(4-amino-3-iodobenzyl)adenosine), S—PIA (S—N⁶-(phenylisopropyl)adenosine), 2-[(2-aminoethyl-aminocarbonylethyl)phenylethyl amino]-5′-N-ethyl-carboxamido adenosine, 2-Cl—IB-MECA (2-chloro-N⁶-(3-iodobenzyl)adenosine-5′-N-methyluronamide), polyadenylic acid, and any mixture thereof.

4. A pharmaceutical composition comprising:
a. a serine protease inhibitor; and
b. adenosine, an adenosine agonist or a pharmaceutically acceptable derivative or prodrug or metabolite thereof.

5. The pharmaceutical composition of claim 4, wherein the serine protease inhibitor is selected from the group consisting of 4-(2-aminoethyl)benzenesulfonylfluoride, ε-amino-n-caproic acid, α₁-antichymotrypsin, antipain, antithrombin III, α₁-antitrypsin, p-amidino phenylmethylsulfonyl fluoride, aprotinin, cathepsin/subtilisin inhibitor (Boc-Val-Phe-NHO-Bz-pCl), chymostatin ([(S)-1-carboxy-2-phenylethyl]-carbamoyl-α-[2-amidohexa hydro-4(S)-pyrimidyl]-(S)-glycyl-[A=Leu, B=Val, or C=Ile]-phenyl alaninal), chymotrypsin inhibitor I, 3,4-dichloroisocoumarin, diisopropylfluorophosphate, dipeptidylpeptidase IV inhibitor I (Ile-Pro-Ile), dipeptidylpeptidase IV inhibitor II (H-Glu-(NHO-Bz)-Pyr), ecotin, elastase inhibitor I (Boc-Ala-Ala-Ala-NHO-Bz), α₂-macroglobulin, PPACK (D-Phe-Pro-Arg-chloromethylketone), PPACK II, N^{a}-tosyl-Lys chloromethyl ketone, N^{a}-tosyl-Phe chloromethyl ketone, and any mixture thereof.

6. The pharmaceutical composition of claim 4, wherein the adenosine agonist or pharmaceutically acceptable derivative is selected from the group consisting of AB-MECA (N⁶-4-aminobenzyl-5′-N-methylcarboxamidoadenosine), CPA (N⁶-cyclopentyladenosine), ADAC (N⁶-[4-[[[4-[[[(2-aminoethyl)amino]carbonyl]methyl]-anilino]carbonyl]methyl]phenyl]adenosine), CCPA (2-chloro-N⁶-cyclopentyl adenosine), CHA (N⁶-cyclohexyladenosine), GR79236 (N⁶-[1S,trans,2-hydroxy cyclopentyl]adenosine), S-ENBA ((2S)—N⁶-(2-endonorbanyl)adenosine), IAB-MECA (N⁶-(4-amino-3-iodobenzyl)adenosine-5′-N-methylcarboxamido adenosine), R—PIA (R—N⁶-(phenylisopropyl)adenosine), ATL146e (4-{3-[6-amino-9-(5-ethyl carbamoyl-3,4-dihydroxy-tetrahydro-furan-2-yl)-9H-purin-2-yl]-prop-2-ynyl}-cyclohexane carboxylic acid methyl ester), CGS-21680 (APEC or 2-[p-(2-carbonyl-ethyl)-phenyl ethyl amino]-5′-N-ethylcarboxamidoadenosine), CV1808 (2-phenylaminoadenosine), HENECA (2-hex-1-ynyl-5′-N-ethylcarboxamido adenosine), NECA (5′-N-ethyl-carboxamido adenosine), PAPA-APEC (2-(4-[2-[(4-aminophenyl)methylcarbonyl]ethyl]phenyl)ethylamino-5′-N-ethyl carboxamidoadenosine), DITC-APEC (2-[p-(4-isothiocyanatophenylamino thiocarbonyl-2-ethyl)-phenylethylamino]-5′-N-ethylcarboxamidoadenosine), DPMA (N⁶-(2(3,5-dimethoxy phenyl)-2-(2-methylphenyl)ethyl)adenosine), S—PHPNECA ((S)-2-phenylhydroxypropynyl-5′-N-ethylcarboxamidoadenosine), WRC-0470 (2-cyclohexyl methylidenehydrazinoadenosine), AMP-579 (1S-[1a,2b,3b,4a(S*)]]-4-[7-[[2-(3-chloro-2-thienyl)-1-methylpropyl]amino]-3H-imidazo[4,5-b]pyridyl-3-yl]cyclopentane carboxamide), IB-MECA (N⁶-(3-iodobenzyl)adenosine-5′-N-methyluronamide), 2-CIADO (2-chloroadenosine), I-ABA (N⁶-(4-amino-3-iodobenzyl)adenosine), S—PIA (S—N⁶-(phenylisopropyl)adenosine), 2-[(2-aminoethyl-aminocarbonylethyl)phenylethyl amino]-5′-N-ethyl-carboxamidoadenosine, 2-Cl—IB-MECA (2-chloro-N⁶-(3-iodobenzyl)adenosine-5′-N-methyluronamide), polyadenylic acid, and any mixture thereof.

7. A method of preventing organ ischemia or reperfusion injury comprising concomitantly administering to a living subject in need thereof
a. a serine protease inhibitor; and
b. adenosine, an adenosine agonist or a pharmaceutically acceptable derivative or prodrug or metabolite thereof.

8. The method of claim 7, wherein the serine protease inhibitor is selected from the group consisting of 4-(2-aminoethyl)benzenesulfonylfluoride, ε-amino-n-caproic acid, α₁-antichymotrypsin, antipain, antithrombin III, α₁-antitrypsin, p-amidinophenylmethyl sulfonyl fluoride, aprotinin, cathepsin/subtilisin inhibitor (Boc-Val-Phe-NHO-Bz-pCl), chymostatin ([(S)-1-carboxy-2-phenylethyl]-carbamoyl-α-[2-amidohexahydro-4-(S)-pyrimidyl]-(S)-glycyl-[A=Leu, B=Val, or C=Ile]-phenylalaninal), chymotrypsin inhibitor I, 3,4-dichloroisocoumarin, diisopropylfluorophosphate, dipeptidylpeptidase IV inhibitor I (Ile-Pro-Ile), dipeptidylpeptidase IV inhibitor II (H-Glu-(NHO-Bz)-Pyr), ecotin, elastase inhibitor I (Boc-Ala-Ala-Ala-NHO-Bz), α₂-macroglobulin PPACK (D-Phe-Pro-Arg-chloromethylketone), PPACK II, N^{a}-tosyl-Lys chloromethyl ketone, N^{a}-tosyl-Phe chloromethyl ketone, and any mixture thereof.

9. The method of claim 7, wherein the adenosine agonist or pharmaceutically acceptable derivative is selected from the group consisting of AB-MECA (N⁶-4-aminobenzyl-5′-N-methylcarboxamidoadenosine), CPA (N⁶-cyclopentyladenosine), ADAC (N⁶-[4-[[[4-[[[(2-aminoethyl)amino]carbonyl]methyl]-anilino]carbonyl]methyl]phenyl]adenosine), CCPA (2-chloro-N⁶-cyclopentyladenosine), CHA (N⁶-cyclohexyladenosine), GR79236 (N⁶-[1S,trans,2-hydroxycyclopentyl]adenosine), S-ENBA ((2S)—N⁶-(2-endonorbanyl)adenosine), IAB-MECA (N⁶-(4-amino-3-iodobenzyl)adenosine-5′-N-methylcarboxamidoadenosine), R—PIA (R—N⁶-(phenylisopropyl)adenosine), ATL146e (4-{3-[6-amino-9-(5-ethylcarbamoyl-3,4-dihydroxy-tetrahydro-furan-2-yl)-9H-purin-2-yl]-prop-2-ynyl}-cyclohexanecarboxylic acid methyl ester), CGS-21680 (APEC or 2-[p-(2-carbonyl-ethyl)-phenyl ethyl amino]-5′-N-ethylcarboxamido adenosine), CV1808 (2-phenylaminoadenosine), HENECA (2-hex-1-ynyl-5′-N-ethylcarboxamido adenosine), NECA (5′-N-ethyl-carboxamido adenosine), PAPA-APEC (2-(4-[2-[(4-amino phenyl)methylcarbonyl]ethyl]phenyl)ethylamino-5′-N-ethyl carboxamidoadenosine), DITC-APEC (2-[p-(4-isothiocyanatophenylamino thiocarbonyl-2-ethyl)-phenylethylamino]-5′-N-ethylcarboxamidoadenosine), DPMA (N⁶-(2(3,5-dimethoxy phenyl)-2-(2-methylphenyl)ethyl)adenosine), S—PHPNECA ((S)-2-phenylhydroxypropynyl-5′-N-ethylcarboxamidoadenosine), WRC-0470 (2-cyclohexylmethylidenehydrazinoadenosine), AMP-579 (1S-[1a,2b,3b,4a(S*)]]-4-[7-[[2-(3-chloro-2-thienyl)-1-methylpropyl]amino]-3H-imidazo[4,5-b]pyridyl-3-yl]cyclo pentane carboxamide), IB-MECA (N⁶-(3-iodobenzyl)adenosine-5′-N-methyluronamide), 2-CIADO (2-chloroadenosine), I-ABA (N⁶-(4-amino-3-iodobenzyl)adenosine), S—PIA (S—N⁶-(phenylisopropyl)adenosine), 2-[(2-aminoethyl-aminocarbonylethyl)phenylethyl amino]-5′-N-ethyl-carboxamidoadenosine, 2-Cl—IB-MECA (2-chloro-N⁶-(3-iodobenzyl)adenosine-5′-N-methyluronamide), polyadenylic acid, and any mixture thereof.

10. A method of preventing organ ischemia or reperfusion injury comprising administering to a living subject in need thereof sequentially in any order
a. a serine protease inhibitor; and
b. adenosine, an adenosine agonist or a pharmaceutically acceptable derivative or prodrug or metabolite thereof.

11. The method of claim 10, wherein the serine protease inhibitor is selected from the group consisting of 4-(2-aminoethyl)benzenesulfonylfluoride, ε-amino-n-caproic acid, α₁-antichymotrypsin, antipain, antithrombin III, α₁-antitrypsin, p-amidinophenylmethyl sulfonyl fluoride, aprotinin, cathepsin/subtilisin inhibitor (Boc-Val-Phe-NHO-Bz-pCl), chymostatin ([(S)-1-carboxy-2-phenylethyl]-carbamoyl-α-[2-amidohexahydro-4(S)-pyrimidyl]-(S)-glycyl-[A=Leu, B=Val or C=Ile]-phenylalaninal), chymotrypsin inhibitor I, 3,4-dichloroisocoumarin, diisopropylfluorophosphate, dipeptidylpeptidase IV inhibitor I (Ile-Pro-Ile), dipeptidylpeptidase IV inhibitor II (H-Glu-(NHO-Bz)-Pyr), ecotin, elastase inhibitor I (Boc-Ala-Ala-Ala-NHO-Bz), α₂-macroglobulin, PPACK (D-Phe-Pro-Arg-chloromethylketone), PPACK II, N^{a}-tosyl-Lys chloromethyl ketone, N^{a}-tosyl-Phe chloromethyl ketone, and any mixture thereof.

12. The method of claim 10, wherein the adenosine agonist or pharmaceutically acceptable derivative is selected from the group consisting of AB-MECA (N⁶-aminobenzyl-5′-N-methylcarboxamidoadenosine), CPA (N⁶-cyclopentyladenosine), ADAC (N⁶-[4-[[[4-[[[(2-aminoethyl)amino]carbonyl]methyl]-anilino]carbonyl]methyl]phenyl]adenosine), CCPA (2-chloro-N⁶-cyclopentyladenosine), CHA (N⁶-cyclohexyladenosine), GR79236 (N⁶-[1S,trans,2-hydroxycyclopentyl]adenosine), S-ENBA ((2S)—N⁶-(2-endonorbanyl)adenosine), IAB-MECA (N⁶-(4-amino-3-iodobenzyl)adenosine-5′-N-methylcarboxamidoadenosine), R—PIA (R—N⁶-(phenylisopropyl)adenosine), ATL146e (4-{3-[6-amino-9-(5-ethylcarbamoyl-3,4-dihydroxy-tetrahydro-furan-2-yl)-9H-purin-2-yl]-prop-2-ynyl}-cyclohexanecarboxylic acid methyl ester), CGS-21680 (APEC or 2-[p-(2-carbonyl-ethyl)-phenyl ethyl amino]-5′-N-ethylcarbox amido adenosine), CV1808 (2-phenylaminoadenosine), HENECA (2-hex-1-ynyl-5′-N-ethylcarboxamido adenosine), NECA (5′-N-ethyl-carboxamido adenosine), PAPA-APEC (2-(4-[2-[(4-amino phenyl)methylcarbonyl]ethyl]phenyl)ethylamino-5′-N-ethyl carboxamidoadenosine), DITC-APEC (2-[p-(4-isothiocyanatophenylamino thiocarbonyl-2-ethyl)-phenylethylamino]-5′-N-ethylcarboxamidoadenosine), DPMA (N⁶-(2(3,5-dimethoxy phenyl)-2-(2-methylphenyl)ethyl)adenosine), S—PHPNECA ((S)-2-phenylhydroxypropynyl-5′-N-ethylcarboxamidoadenosine), WRC-0470 (2-cyclohexylmethylidenehydrazinoadenosine), AMP-579 (1S-[1a,2b,3b,4a(S*)]]-4-[7-[[2-(3-chloro-2-thienyl)-1-methylpropyl]amino]-3H-imidazo[4,5-b]pyridyl-3-yl]cyclo pentane carboxamide), IB-MECA (N⁶-(3-iodobenzyl)adenosine-5′-N-methyluronamide), 2-CIADO (2-chloroadenosine), I-ABA (N⁶-(4-amino-3-iodobenzyl)adenosine), S—PIA (S—N⁶-(phenylisopropyl)adenosine), 2-[(2-aminoethyl-aminocarbonylethyl)phenylethyl amino]-5′-N-ethyl-carboxamidoadenosine, 2-Cl—IB-MECA (2-chloro-N⁶-(3-iodobenzyl)adenosine-5′-N-methyluronamide), polyadenylic acid, and any mixture thereof.

13. A method of preventing organ or tissue injury at a predetermined point or period of intervention comprising administrating to a living subject in need thereof a pharmaceutical composition comprising:
a. a serine protease inhibitor; and
b. adenosine, an adenosine agonist or a pharmaceutically acceptable derivative or prodrug or metabolite thereof.

14. The method of claim 13, wherein the organ or tissue injury is related to at least one of cardiac surgery, non-surgical cardiac revascularization, organ transplantation, perfusion, ischemia, reperfusion, ischemia-reperfusion injury, oxidant injury, cytokine induced injury, shock induced injury, resuscitations injury, and apoptosis.

15. The method of claim 13, wherein the administrating is taken at the predetermined point of intervention related to at least one of pre-treatment regimen, pharmacological preconditioning, reperfusion, or post interventional therapy, wherein the pharmacological preconditioning is a treatment administered before the ischemic intervention followed by a brief period of reperfusion or washout.

16. The method of claim 13, wherein the serine protease inhibitor is selected from the group consisting of 4-(2-aminoethyl)benzenesulfonylfluoride, ε-amino-n-caproic acid, α₁-antichymotrypsin, antipain, antithrombin III, α₁-antitrypsin, p-amidinophenylmethyl sulfonyl fluoride, aprotinin, cathepsin/subtilisin inhibitor (Boc-Val-Phe-NHO-Bz-pCl), chymostatin ([(S)-1-carboxy-2-phenylethyl]-carbamoyl-α-[2-amidohexahydro-4(S)-pyrimidyl]-(S)-glycyl-[A=Leu, B=Val or C=Ile]-phenylalaninal), chymotrypsin inhibitor I, 3,4-dichloroisocoumarin, diisopropylfluorophosphate, dipeptidylpeptidase IV inhibitor I (Ile-Pro-Ile), dipeptidylpeptidase IV inhibitor II (H-Glu-(NHO-Bz)-Pyr), ecotin, elastase inhibitor I (Boc-Ala-Ala-Ala-NHO-Bz), α₂-macroglobulin, PPACK (D-Phe-Pro-Arg-chloromethylketone), PPACK II, N^{a}-tosyl-Lys chloromethyl ketone, N^{a}-tosyl-Phe chloromethyl ketone, and any mixture thereof.

17. The method of claim 13, wherein the adenosine agonist or pharmaceutically acceptable derivative is selected from the group consisting of AB-MECA (N⁶-4-aminobenzyl-5′-N-methylcarboxamidoadenosine), CPA (N⁶-cyclopentyladenosine), ADAC (N⁶-[4-[[[4-[[[(2-aminoethyl)amino]carbonyl]methyl]-anilino]carbonyl]methyl]phenyl]adenosine), CCPA (2-chloro-N⁶-cyclopentyladenosine), CHA (N⁶-cyclohexyladenosine), GR79236 (N⁶-[1S,trans,2-hydroxycyclopentyl]adenosine), S-ENBA ((2S)—N⁶-(2-endonorbanyl)adenosine), IAB-MECA (N⁶-(4-amino-3-iodobenzyl)adenosine-5′-N-methylcarboxamidoadenosine), R—PIA (R—N⁶-(phenylisopropyl)adenosine), ATL146e (4-{3-[6-amino-9-(5-ethylcarbamoyl-3,4 -dihydroxy-tetrahydro-furan-2-yl)-9H-purin-2-yl]-prop-2-ynyl}-cyclohexanecarboxylic acid methyl ester), CGS-21680 (APEC or 2-[p-(2-carbonyl-ethyl)-phenyl ethyl amino]-5′-N-ethylcarboxamidoadenosine), CV1808 (2-phenylaminoadenosine), HENECA (2-hex-1-ynyl-5′-N-ethylcarboxamido adenosine), NECA (5′-N-ethyl-carboxamido adenosine), PAPA-APEC (2-(4-[2-[(4-aminophenyl)methyl carbonyl]ethyl]phenyl)ethylamino-5′-N-ethyl carboxamidoadenosine), DITC-APEC (2-[p-(4-isothiocyanatophenylamino thiocarbonyl-2-ethyl)-phenylethylamino]-5′-N-ethylcarboxamidoadenosine), DPMA (N⁶-(2(3,5-dimethoxy phenyl)-2-(2-methyl phenyl)ethyl)adenosine), S—PHPNECA ((S)-2-phenylhydroxypropynyl-5′-N-ethyl carboxamidoadenosine), WRC-0470 (2-cyclohexylmethylidenehydrazinoadenosine), AMP-579 (1S-[1a,2b,3b,4a(S*)]]-4-[7-[[2-(3-chloro-2-thienyl)-1-methylpropyl]amino]-3H-imidazo[4,5-b]pyridyl-3-yl]cyclopentane carboxamide), IB-MECA (N⁶-(3-iodobenzyl)adenosine-5′-N-methyluronamide), 2-CIADO (2-chloroadenosine), I-ABA (N⁶-(4-amino-3-iodobenzyl)adenosine), S—PIA (S—N⁶-(phenylisopropyl)adenosine), 2-[(2-amino ethyl-aminocarbonylethyl)phenylethyl amino]-5′-N-ethyl-carboxamidoadenosine, 2-Cl—IB-MECA (2-chloro-N⁶-(3-iodobenzyl)adenosine-5′-N-methyluronamide), polyadenylic acid, and any mixture thereof.

18. A method of preventing organ ischemia or reperfusion injury comprising administrating to a living subject in need thereof a pharmaceutical composition comprising:
a. a protease inhibitor; and
b. an agent that alters activities of G protein coupled receptors and cAMP, an analog or a pharmaceutically acceptable derivative or prodrug or metabolite thereof.

19. The method of claim 18, wherein the protease inhibitor is selected from the group consisting of 4-(2-aminoethyl)benzenesulfonylfluoride, ε-amino-n-caproic acid, α₁-antichymotrypsin, antipain, antithrombin III, α₁-antitrypsin, p-amidinophenylmethyl sulfonyl fluoride, aprotinin, cathepsin/subtilisin inhibitor (Boc-Val-Phe-NHO-Bz-pCl), chymostatin ([(S)-1-carboxy-2-phenylethyl]-carbamoyl-α-[2-amidohexahydro-4(S)-pyrimidyl]-(S)-glycyl-[A=Leu, B=Val, or C=Ile]-phenylalaninal), chymotrypsin inhibitor I, 3,4-dichloroisocoumarin, diisopropylfluoro phosphate, dipeptidylpeptidase IV inhibitor I (Ile-Pro-Ile), dipeptidylpeptidase IV inhibitor II (H-Glu-(NHO-Bz)-Pyr), ecotin, elastase inhibitor I (Boc-Ala-Ala-Ala-NHO-Bz), α2-macroglobulin PPACK (D-Phe-Pro-Arg-chloromethylketone), PPACK II, N^{a}-tosyl-Lys chloromethyl ketone, N^{a}-tosyl-Phe chloromethyl ketone, acetyl-pepstatin (Ac-Val-Val-(3S,4S)-Sta-Ala-(3S,4S)-Sta-OH), calpain inhibitor I (N-acetyl-Leu-Leu-norleucinal), calpain inhibitor II (N-acetyl-Leu-Leu-Met-CHO), amastatin ([(2S,2R)]-3-amino-2-hydroxy-5-methylhexanoyl]-Val-Val-Asp-OH), arphamenine A ((2R,5S)-5-amino-8-guanidino-4-oxo-2-phenylmethyl octanoic acid), arphamenine B ((2R,5S)-5-amino-8-guanidino-4-oxo-2-p-hydroxyphenyl methyloctanoic acid), benzamidine, bestatin ([(2S,2R)-3-amino-2-hydroxy-4-phenyl butanoyl]-L-Leucine), CA-074 ((L-3-trans-[propylcarbamoyl]oxirane-2-carbonyl)-L-isoleucyl-L-proline), CA-074-Me ((L-3-trans-[propylcarbamoyl]oxirane-2-carbonyl)-L-isoleucyl-L-proline-methylester), calpastatin, calpeptin (benzyloxycarbonylleucyl-norleucinal), carboxypeptidase inhibitor, cathepsin inhibitor I (Z-Phe-Gly-NHO-Bz), cathepsin inhibitor II (Z-Phe-Gly-NHO-Bz-pMe), cathepsin inhibitor III (Z-Phe-Gly-NHO-Bz-pOMe), cathepsin B inhibitor I (Z-Phe-Ala-CH₂F), cathepsin B inhibitor II (Ac-Leu-Val-lysinal), cathepsin L inhibitor I (Z-Phe-Phe-CH₂F), cathepsin L inhibitor II (Z-Phe-Tyr-CHO), cathepsin L inhibitor III (Z-Phe-Tyr-(t-Bu)-CHN₂), cathepsin L inhibitor IV (1-naphthalenesulfonyl-Ile-Trp-CHO), cathepsin L inhibitor V (Z-Phe-Tyr(OtBu)-COCHO), cathepsin L inhibitor VI N-(4-biphenylacetyl)-S-methylcysteine-(D)-Arg-Phe-β-phenethylamide), cathepsin S inhibitor (Z-Phe-Leu-COCHO), cystatin, diprotin A (H-Ile-Pro-Ile-OH), E-64 (trans-epoxysuccinyl-L-leucylamido-(4-guanidino)butane), E-64 d (loxistatin, or (2S,3S)-trans-epoxysuccinyl-L-leucylamido-3-methylbutane ethyl ester), ebelactone A (3,11-dihydroxy-2,4,6,8,10,12-hexamethyl-9-oxo-6-tetradecenoic 1,3-lactone), ebelactone B (2-ethyl-3,11-dihydroxy-4,6,8,10,12-penta methyl-9-oxo-6-tetradecenoic 1,3-lactone), EDTA (ethylenediamine tetraacetic acid), EGTA (ethyleneglycol-bis(β-aminoethyl)-N,N,N′,N′-tetraacetic acid), elastase inhibitor II (MeOSuc-Ala-Ala-Pro-Ala-CMK), elastase inhibitor III (MeOSuc-Ala-Ala-Pro-Val-CMK), elastatinal (Leu-(Cap)-Gln-Ala-al or N—[(S)-1-carboxy-isopentyl)-carbamoyl-alpha-(2-iminohexahydro-4(S)-pyrimidyl]-L-glycyl-L-glutaminyl-L-alaninal), E-64 (trans-epoxysuccinyl-L-leucylamido-(4-guanidino)butane), E-64 d (loxistatin, or (2S,3S)-trans-epoxysuccinyl-L-leucylamido-3-methylbutane ethyl ester), N-ethyl maleimide, GGACK (1,5-dansyl-L-glutamyl-L-glycyl-L-arginine chloro methyl ketone), galardin (N—[(2S)-(methoxycarbonylmethyl)-4-methylpentanoyl]-L-tryptophan-methyl amide), 2-guanidinoethylmercaptosuccinic acid, hirudin, HIV protease inhibitor (Ac-Leu-Val-phenylalaninal), leuhistin (((2R,3S)-3-amino-2-hydroxy-2-(1H-imidazol-4-ylmethyl)-5-methyl)-5-methylhexanoic acid), leupeptin (acetyl-leucyl-leucyl-arginal), NCO-700, PEFABLOC SC (4-(2-aminoethyl)-benzenesulfonyl fluoride), pepstatin (isovaleryl-Val-Val-4-amino-3-hydroxy-6-methylheptanoyl-Ala-4-amino-3-hydroxy-6-methylheptanoic acid), phebestin ((2S,3R)-3-amino-2-hydroxy-4-phenylbutanoyl-L-valyl-L-phenylalanine), PMSF (phenyl methyl sulfonyl fluoride), phosphoramidon (N-alpha-L-rhamnopyranosyloxy(hydroxyl phosphinyl)-L-Leucyl-L-tryptophan, plummer's inhibitor (D,L-2-mercaptomethyl-3-guanidino-ethylthiopropanoic acid), 1,10-phenanthroline, subtilisin inhibitor I (Boc-Ala-Ala-NHO-Bz), subtilisin inhibitor II (Z-Gly-Phe-NHO-Bz), subtilisin inhibitor III (Z-Gly-Phe-NHO-Bz-pOMe), subtilisin inhibitor IV (Boc-Pro-Phe-NHO-Bz-pCl), subtilisin inhibitor V (Boc-Ala-Pro-Phe-NHO-Bz), TIMP-2 (tissue inhibitor of metalloproteinase 2), trypsin inhibitor, secretory leukocyte protease inhibitor, and any mixture there of.

20. The method of claim 18, wherein the agent that alters activities of G protein coupled receptors and cAMP or pharmaceutically acceptable derivative is selected from the group consisting of AB-MECA (N⁶-4-aminobenzyl-5′-N-methylcarboxamidoadenosine), CPA (N⁶-cyclopentyladenosine), ADAC (N⁶-[4-[[[4-[[[(2-aminoethyl)amino]carbonyl]methyl]-anilino]carbonyl]methyl]phenyl]adenosine), CCPA (2-chloro-N⁶-cyclopentyl adenosine), CHA (N⁶-cyclohexyladenosine), GR79236 (N⁶-[1S,trans,2-hydroxycyclo pentyl]adenosine), S-ENBA ((2S)—N⁶-(2-endonorbanyl)adenosine), IAB-MECA (N⁶-(4-amino-3-iodobenzyl)adenosine-5′-N-methylcarboxamidoadenosine), R—PIA (R—N⁶-(phenyl isopropyl)adenosine), ATL146e (4-{3-[6-amino-9-(5-ethylcarbamoyl-3,4-dihydroxy-tetrahydro-furan-2-yl)-9H-purin-2-yl]-prop-2-ynyl}-cyclohexanecarboxylic acid methyl ester), CGS-21680 (APEC or 2-[p-(2-carbonyl-ethyl)-phenyl ethyl amino]-5′-N-ethylcarboxamidoadenosine), CV1808 (2-phenylaminoadenosine), HENECA (2-hex-1-ynyl-5′-N-ethylcarboxamido adenosine), NECA (5′-N-ethyl-carboxamido adenosine), PAPA-APEC (2-(4-[2-[(4-aminophenyl)methylcarbonyl]ethyl]phenyl)ethylamino-5′-N-ethyl carboxamidoadenosine), DITC-APEC (2-[p-(4-isothiocyanatophenylamino thio carbonyl-2-ethyl)-phenylethylamino]-5′-N-ethylcarboxamidoadenosine), DPMA (N⁶-(2(3,5-dimethoxy phenyl)-2-(2-methylphenyl)ethyl)adenosine), S—PHPNECA ((S)-2-phenylhydroxypropynyl-5′-N-ethylcarboxamidoadenosine), WRC-0470 (2-cyclohexyl methylidenehydrazinoadenosine), AMP-579 (1S-[1a,2b,3b,4a(S*)]]-4-[7-[[2-(3-chloro-2-thienyl)-1-methylpropyl]amino]-3H-imidazo[4,5-b]pyridyl-3-yl]cyclopentane carboxamide), IB-MECA (N⁶-(3-iodobenzyl)adenosine-5′-N-methyluronamide), 2-CIADO (2-chloroadenosine), I-ABA (N⁶-(4-amino-3-iodobenzyl)adenosine), S—PIA (S—N⁶-(phenylisopropyl)adenosine), 2-[(2-aminoethyl-aminocarbonylethyl)phenylethyl amino]-5′-N-ethyl-carboxamidoadenosine, 2-Cl—IB-MECA (2-chloro-N⁶-(3-iodobenzyl)adenosine-5′-N-methyluronamide), adenosine, polyadenylic acid, and any mixture thereof.

21. A pharmaceutical composition comprising:
a. a protease inhibitor; and
b. an agent that alters activities of G protein coupled receptors and cAMP or a pharmaceutically acceptable derivative or prodrug thereof.

22. The pharmaceutical composition of claim 21, wherein the protease inhibitor is selected from the group consisting of 4-(2-aminoethyl)benzenesulfonylfluoride, ε-amino-n-caproic acid, α₁-antichymotrypsin, antipain, antithrombin III, α₁-antitrypsin, p-amidinophenylmethyl sulfonyl fluoride, aprotinin, cathepsin/subtilisin inhibitor (Boc-Val-Phe-NHO-Bz-pCl), chymostatin ([(S)-1-carboxy-2-phenylethyl]-carbamoyl-α-[2-amidohexahydro-4(S)-pyrimidyl]-(S)-glycyl-[A=Leu, B=Val, or C=Ile]-phenylalaninal), chymotrypsin inhibitor I, 3,4-dichloroisocoumarin, diisopropylfluoro phosphate, dipeptidylpeptidase IV inhibitor I (Ile-Pro-Ile), dipeptidylpeptidase IV inhibitor II (H-Glu-(NHO-Bz)-Pyr), ecotin, elastase inhibitor I (Boc-Ala-Ala-Ala-NHO-Bz), α₂-macroglobulin, PPACK (D-Phe-Pro-Arg-chloromethylketone), PPACK II, N^{a}-tosyl-Lys chloromethyl ketone, N^{a}-tosyl-Phe chloromethyl ketone, acetyl-pepstatin (Ac-Val-Val-(3S,4S)-Sta-Ala-(3S,4S)-Sta-OH), calpain inhibitor I (N-acetyl-Leu-Leu-norleucinal), calpain inhibitor II (N-acetyl-Leu-Leu-Met-CHO), amastatin ([(2S,2R)]-3-amino-2-hydroxy-5-methylhexanoyl]-Val-Val-Asp-OH), arphamenine A ((2R,5S)-5-amino-8-guanidino-4-oxo-2-phenylmethyl octanoic acid), arphamenine B ((2R,5S)-5-amino-8-guanidino-4-oxo-2-p-hydroxyphenyl methyloctanoic acid), benzamidine, bestatin ([(2S, 2R)-3-amino-2-hydroxy-4-phenyl butanoyl]-L-Leucine), CA-074 ((L-3-trans-[propylcarbamoyl]oxirane-2-carbonyl)-L-isoleucyl-L-proline), CA-074-Me ((L-3-trans-[propylcarbamoyl]oxirane-2-carbonyl)-L-isoleucyl-L-proline-methylester), calpastatin, calpeptin (benzyloxycarbonylleucyl-norleucinal), carboxypeptidase inhibitor, cathepsin inhibitor I (Z-Phe-Gly-NHO-Bz), cathepsin inhibitor II (Z-Phe-Gly-NHO-Bz-pMe), cathepsin inhibitor III (Z-Phe-Gly-NHO-Bz-pOMe), cathepsin B inhibitor I (Z-Phe-Ala-CH₂F), cathepsin B inhibitor II (Ac-Leu-Val-lysinal), cathepsin L inhibitor I (Z-Phe-Phe-CH₂F), cathepsin L inhibitor II (Z-Phe-Tyr-CHO), cathepsin L inhibitor III (Z-Phe-Tyr-(t-Bu)-CHN₂), cathepsin L inhibitor IV (1-naphthalenesulfonyl-Ile-Trp-CHO), cathepsin L inhibitor V (Z-Phe-Tyr(OtBu)-COCHO), cathepsin L inhibitor VI (N-(4-biphenylacetyl)-S-methylcysteine-(D)-Arg-Phe-β-phenethylamide), cathepsin S inhibitor (Z-Phe-Leu-COCHO), cystatin, diprotin A (H-Ile-Pro-Ile-OH), E-64 (trans-epoxysuccinyl-L-leucylamido-(4-guanidino)butane), E-64 d (loxistatin, or (2S,3S)-trans-epoxysuccinyl-L-leucylamido-3-methylbutane ethyl ester), ebelactone A (3,11-dihydroxy-2,4,6,8,10,12-hexamethyl-9-oxo-6-tetradecenoic 1,3-lactone), ebelactone B (2-ethyl-3,11-dihydroxy-4,6,8,10,12-penta methyl-9-oxo-6-tetradecenoic 1,3-lactone), EDTA (ethylenediamine tetraacetic acid), EGTA (ethyleneglycol-bis(β-aminoethyl)-N,N,N′,N′-tetraacetic acid), elastase inhibitor II (MeOSuc-Ala-Ala-Pro-Ala-CMK), elastase inhibitor III (MeOSuc-Ala-Ala-Pro-Val-CMK), elastatinal (Leu-(Cap)-Gln-Ala-al or N—[(S)-1-carboxy-isopentyl)-carbamoyl-alpha-(2-iminohexahydro-4(S)-pyrimidyl]-L-glycyl-L-glutaminyl-L-alaninal), E-64 (trans-epoxysuccinyl-L-leucylamido-(4-guanidino)butane), E-64d (loxistatin, or (2S,3S)-trans-epoxysuccinyl-L-leucylamido-3-methylbutane ethyl ester), N-ethyl maleimide, GGACK (1,5-dansyl-L-glutamyl-L-glycyl-L-arginine chloro methyl ketone), galardin (N-[(2S)-(methoxycarbonylmethyl)-4-methylpentanoyl]-L-tryptophan-methyl amide), 2-guanidinoethylmercaptosuccinic acid, hirudin, HIV protease inhibitor (Ac-Leu-Val-phenylalaninal), leuhistin (((2R,3S)-3-amino-2-hydroxy-2-(1H-imidazol-4-ylmethyl)-5-methyl)-5-methylhexanoic acid), leupeptin (acetyl-leucyl-leucyl-arginal), NCO-700, PEFABLOC SC (4-(2-aminoethyl)-benzenesulfonyl fluoride), pepstatin (isovaleryl-Val-Val-4-amino-3-hydroxy-6-methylheptanoyl-Ala-4-amino-3-hydroxy-6-methylheptanoic acid), phebestin ((2S,3R)-3-amino-2-hydroxy-4-phenylbutanoyl-L-valyl-L-phenylalanine), PMSF (phenyl methyl sulfonyl fluoride), phosphoramidon (N-alpha-L-rhamnopyranosyloxy(hydroxyl phosphinyl)-L-Leucyl-L-tryptophan, plummer's inhibitor (D,L-2-mercaptomethyl-3-guanidino-ethylthiopropanoic acid), 1,10-phenanthroline, subtilisin inhibitor I (Boc-Ala-Ala-NHO-Bz), subtilisin inhibitor II (Z-Gly-Phe-NHO-Bz), subtilisin inhibitor III (Z-Gly-Phe-NHO-Bz-pOMe), subtilisin inhibitor IV (Boc-Pro-Phe-NHO-Bz-pCl), subtilisin inhibitor V (Boc-Ala-Pro-Phe-NHO-Bz), TIMP-2 (tissue inhibitor of metalloproteinase 2), trypsin inhibitor, secretory leukocyte protease inhibitor, and any mixture there of.

23. The pharmaceutical composition of claim 21, wherein the agent that alters activities of G protein coupled receptors and cAMP or pharmaceutically acceptable derivative is selected from the group consisting of AB-MECA (N⁶-4-aminobenzyl-5′-N-methylcarbox amidoadenosine), CPA (N⁶-cyclopentyladenosine), ADAC (N⁶-[4-[[[4-[[[(2-aminoethyl)amino]carbonyl]methyl]-anilino]carbonyl]methyl]phenyl]adenosine), CCPA (2-chloro-N⁶-cyclopentyladenosine), CHA (N⁶-cyclohexyladenosine), GR79236 (N⁶-[1S,trans,2-hydroxycyclopentyl]adenosine), S-ENBA ((2S)—N⁶-(2-endonorbanyl)adenosine), IAB-MECA (N⁶-(4-amino-3-iodobenzyl)adenosine-5′-N-methylcarboxamidoadenosine), R—PIA (R—N⁶-(phenylisopropyl)adenosine), ATL146e (4-{3-[6-amino-9-(5-ethyl carbamoyl-3,4-dihydroxy-tetrahydro-furan-2-yl)-9H-purin-2-yl]-prop-2-ynyl}-cyclohexanecarboxylic acid methyl ester), CGS-21680 (APEC or 2-[p-(2-carbonyl-ethyl)-phenyl ethyl amino]-5′-N-ethylcarboxamidoadenosine), CV1808 (2-phenylamino adenosine, HENECA (2-hex-1-ynyl-5′-N-ethylcarboxamido adenosine), NECA (5′-N-ethyl-carboxamido adenosine), PAPA-APEC (2-(4-[2-[(4-aminophenyl)methylcarbonyl]ethyl]phenyl)ethylamino-5′-N-ethyl carboxamidoadenosine), DITC-APEC (2-[p-(4-isothiocyanatophenylamino thiocarbonyl-2-ethyl)-phenylethylamino]-5′-N-ethylcarbox amido adenosine), DPMA (N⁶-(2(3,5-dimethoxy phenyl)-2-(2-methylphenyl)ethyl)adenosine), S—PHPNECA ((S)-2-phenylhydroxypropynyl-5′-N-ethylcarboxamido adenosine), WRC-0470 (2-cyclohexylmethylidenehydrazinoadenosine), AMP-579 (1S-[1a,2b,3b,4a(S*)]]-4-[7-[[2-(3-chloro-2-thienyl)-1-methylpropyl]amino]-3H-imidazo[4,5-b) pyridyl-3-yl]cyclopentane carboxamide), IB-MECA (N⁶-(3-iodobenzyl)adenosine-5′-N-methyluronamide), 2-CIADO (2-chloroadenosine), I-ABA (N⁶-(4-amino-3-iodobenzyl)adenosine), S—PIA (S—N⁶-(phenylisopropyl)adenosine), 2-[(2-aminoethyl-aminocarbonylethyl)phenylethyl amino]-5′-N-ethyl-carboxamidoadenosine, 2-Cl—IB-MECA (2-chloro-N⁶-(3-iodobenzyl)adenosine-5′-N-methyluronamide), adenosine, polyadenylic acid, and any mixture thereof.

24. A method of preventing organ ischemia or reperfusion injury comprising concomitantly administering to a living subject in need thereof
a. a protease inhibitor; and
b. an agent that alters activities of G protein coupled receptors and cAMP or a pharmaceutically acceptable derivative or prodrug thereof.

25. The method of claim 24, wherein the protease inhibitor is selected from the group consisting of 4-(2-aminoethyl)benzenesulfonylfluoride, ε-acid-n-caproic acid, α₁-antichymotrypsin, antipain, antithrombin III, α₁-antitrypsin, p-amidinophenylmethyl sulfonyl fluoride, aprotinin, cathepsin/subtilisin inhibitor (Boc-Val-Phe-NHO-Bz-pCl), chymostatin ([(S)-1-carboxy-2-phenylethyl]-carbamoyl-α-[2-amidohexahydro-4(S)-pyrimidyl]-(S)-glcyl-[A=Leu, B=Val, or C=Ile]-phenylalaninal), chymotrypsin inhibitor I, 3,4-dichloroisocoumarin, diisopropylfluoro phosphate, dipeptidylpeptidase IV inhibitor I (Ile-Pro-Ile), dipeptidylpeptidase IV inhibitor II (H-Glu-(NHO-Bz)-Pyr), ecotin, elastase inhibitor I (Boc-Ala-Ala-Ala-NHO-Bz), α2-macroglobulin, PPACK (D-Phe-Pro-Arg-chloromethylketone), PPACK II, N^{a}-tosyl-Lys chloromethyl ketone, N^{a}-tosyl-Phe chloromethyl ketone, acetyl-pepstatin (Ac-Val-Val-(3S,4S)-Sta-Ala-(3S,4S)-Sta-OH), calpain inhibitor I (N-acetyl-Leu-Leu-norleucinal), calpain inhibitor II (N-acetyl-Leu-Leu-Met-CHO), amastatin ([(2S,2R)]-3-amino-2-hydroxy-5-methylhexanoyl]-Val-Val-Asp-OH), arphamenine A ((2R,5S)-5-amino-8-guanidino-4-oxo-2-phenylmethyl octanoic acid), arphamenine B ((2R,5S)-5-amino-8-guanidino-4-oxo-2-p-hydroxyphenyl methyloctanoic acid), benzamidine, bestatin ([(2S,2R)-3-amino-2-hydroxy-4-phenyl butanoyl]-L-Leucine), CA-074 ((L-3-trans-[propylcarbamoyl]oxirane-2-carbonyl)-L-isoleucyl-L-proline), CA-074-Me ((L-3-trans-[propylcarbamoyl]oxirane-2-carbonyl)-L-isoleucyl-L-proline-methylester), calpastatin, calpeptin (benzyloxycarbonylleucyl-norleucinal), carboxypeptidase inhibitor, cathepsin inhibitor I (Z-Phe-Gly-NHO-Bz), cathepsin inhibitor II (Z-Phe-Gly-NHO-Bz-pMe), cathepsin inhibitor III (Z-Phe-Gly-NHO-Bz-pOMe), cathepsin B inhibitor I (Z-Phe-Ala-CH₂F), cathepsin B inhibitor II (Ac-Leu-Val-lysinal), cathepsin L inhibitor I (Z-Phe-Phe-CH₂F), cathepsin L inhibitor II (Z-Phe-Tyr-CHO), cathepsin L inhibitor III (Z-Phe-Tyr-(t-Bu)-CHN₂), cathepsin L inhibitor IV (1-naphthalenesulfonyl-Ile-Trp-CHO), cathepsin L inhibitor V (Z-Phe-Tyr(OtBu)-COCHO), cathepsin L inhibitor VI (N-(4-biphenylacetyl)-S-methylcysteine-(D)-Arg-Phe-β-phenethylamide), cathepsin S inhibitor (Z-Phe-Leu-COCHO), cystatin, diprotin A (H-Ile-Pro-Ile-OH), E-64 (trans-epoxysuccinyl-L-leucylamido-(4-guanidino)butane), E-64 d (loxistatin, or (2S,3S)-trans-epoxysuccinyl-L-leucylamido-3-methylbutane ethyl ester), ebelactone A (3,11-dihydroxy-2,4,6,8,10,12-hexamethyl-9-oxo-6-tetradecenoic 1,3-lactone), ebelactone B (2-ethyl-3,11-dihydroxy-4,6,8,10,12-penta methyl-9-oxo-6-tetradecenoic 1,3-lactone), EDTA (ethylenediamine tetraacetic acid), EGTA (ethyleneglycol-bis(β-aminoethyl)-N,N,N′,N′-tetraacetic acid), elastase inhibitor II (MeOSuc-Ala-Ala-Pro-Ala-CMK), elastase inhibitor III (MeOSuc-Ala-Ala-Pro-Val-CMK), elastatinal (Leu-(Cap)-Gln-Ala-al or N—[(S)-1-carboxy-isopentyl)-carbamoyl-alpha-(2-iminohexahydro-4(S)-pyrimidyl]-L-glycyl-L-glutaminyl-L-alaninal), E-64 (trans-epoxysuccinyl-L-leucylamido-(4-guanidino)butane), E-64d (loxistatin, or (2S,3S)-trans-epoxysuccinyl-L-leucylamido-3-methylbutane ethyl ester), N-ethyl maleimide, GGACK (1,5-dansyl-L-glutamyl-L-glycyl-L-arginine chloro methyl ketone), galardin (N-[(2S)-(methoxycarbonylmethyl)-4-methylpentanoyl]-L-tryptophan-methyl amide), 2-guanidinoethylmercaptosuccinic acid, hirudin, HIV protease inhibitor (Ac-Leu-Val-phenylalaninal), leuhistin (((2R,3S)-3-amino-2-hydroxy-2-(1H-imidazol-4-ylmethyl)-5-methyl)-5-methylhexanoic acid), leupeptin (acetyl-leucyl-leucyl-arginal), NCO-700, PEFABLOC SC (4-(2-aminoethyl)-benzenesulfonyl fluoride), pepstatin (isovaleryl-Val-Val-4-amino-3-hydroxy-6-methylheptanoyl-Ala-4-amino-3-hydroxy-6-methylheptanoic acid), phebestin ((2S,3R)-3-amino-2-hydroxy-4-phenylbutanoyl-L-valyl-L-phenylalanine), PMSF (phenyl methyl sulfonyl fluoride), phosphoramidon (N-alpha-L-rhamnopyranosyloxy(hydroxyl phosphinyl)-L-Leucyl-L-tryptophan, plummer's inhibitor (D,L-2-mercaptomethyl-3-guanidino-ethylthiopropanoic acid), 1,10-phenanthroline, subtilisin inhibitor I (Boc-Ala-Ala-NHO-Bz), subtilisin inhibitor II (Z-Gly-Phe-NHO-Bz), subtilisin inhibitor III (Z-Gly-Phe-NHO-Bz-pOMe), subtilisin inhibitor IV (Boc-Pro-Phe-NHO-Bz-pCl), subtilisin inhibitor V (Boc-Ala-Pro-Phe-NHO-Bz), TIMP-2 (tissue inhibitor of metalloproteinase 2), trypsin inhibitor, secretory leukocyte protease inhibitor, and any mixture there of.

26. The method of claim 24, wherein the agent that alters the activities of G-protein coupled receptors and cAMP or pharmaceutically acceptable derivative is selected from the group consisting of AB-MECA (N⁶-4-aminobenzyl-5′-N-methylcarboxamidoadenosine), CPA (N⁶-cyclopentyladenosine), ADAC (N⁶-[4-[[[4-[[[(2-aminoethyl)amino]carbonyl]methyl]-anilino]carbonyl]methyl]phenyl]adenosine), CCPA (2-chloro-N⁶-cyclopentyl adenosine), CHA (N⁶-cyclohexyladenosine), GR79236 (N⁶-[1S,trans,2-hydroxycyclo pentyl]adenosine), S-ENBA ((2S)-N-(2-endonorbanyl)adenosine), IAB-MECA (N⁶-(4-amino-3-iodobenzyl)adenosine-5′-N-methylcarboxamidoadenosine), R—PIA (R—N⁶-(phenyl isopropyl)adenosine), ATL146e (4-{3-[6-amino-9-(5-ethylcarbamoyl-3,4-dihydroxy-tetrahydro-furan-2-yl)-9H-purin-2-yl]-prop-2-ynyl}-cyclohexanecarboxylic acid methyl ester), CGS-21680 (APEC or 2-[p-(2-carbonyl-ethyl)-phenyl ethyl amino]-5′-N-ethylcarboxamidoadenosine), CV1808 (2-phenylaminoadenosine), HENECA (2-hex-1-ynyl-5′-N-ethylcarboxamido adenosine), NECA (5′-N-ethyl-carboxamido adenosine), PAPA-APEC (2-(4-[2-[(4-aminophenyl)methylcarbonyl]ethyl]phenyl)ethylamino-5′-N-ethyl carboxamidoadenosine), DITC-APEC (2-[p-(4-isothiocyanatophenylamino thio carbonyl-2-ethyl)-phenylethylamino]-5′-N-ethylcarboxamidoadenosine), DPMA (N⁶-(2(3,5-dimethoxy phenyl)-2-(2-methylphenyl)ethyl)adenosine), S—PHPNECA ((S)-2-phenylhydroxypropynyl-5′-N-ethylcarboxamidoadenosine), WRC-0470 (2-cyclohexyl methylidenehydrazinoadenosine), AMP-579 (1S-[1a,2b,3b,4a(S*)]]-4-[7-[[2-(3-chloro-2-thienyl)-1-methylpropyl]amino]-3H-imidazo[4,5-b]pyridyl-3-yl]cyclopentane carbox amide), IB-MECA (N⁶-(3-iodobenzyl)adenosine-5′-N-methyluronamide), 2-CIADO (2-chloroadenosine), I-ABA (N⁶-(4-amino-3-iodobenzyl)adenosine), S—PIA (S—N⁶-(phenyl isopropyl)adenosine), 2-[(2-aminoethyl-aminocarbonylethyl)phenylethyl amino]-5′-N-ethyl-carboxamidoadenosine, 2-Cl—IB-MECA (2-chloro-N⁶-(3-iodobenzyl)adenosine-5′-N-methyluronamide), adenosine, polyadenylic acid, and any mixture thereof.

27. A method of preventing organ ischemia or reperfusion injury comprising administering to a living subject in need thereof sequentially in any order
a. a protease inhibitor; and
b. an agent that alters activities of G protein coupled receptors and cAMP or a pharmaceutically acceptable derivative or prodrug thereof.

28. The method of claim 27, wherein the serine protease inhibitor is selected from the group consisting of 4-(2-aminoethyl)benzenesulfonylfluoride, ε-amino-n-caproic acid, α1-antichymotrypsin, antipain, antithrombin III, α1-antitrypsin, p-amidinophenylmethyl sulfonyl fluoride, aprotinin, cathepsin/subtilisin inhibitor (Boc-Val-Phe-NHO-Bz-pCl), chymostatin ([S)-1-carboxy-2-phenylethyl]-carbamoyl-α-[2-amidohexahydro-4(S)-pyrimidyl]-(S)-glycyl-[A=Leu, B=Val or C=Ile]-phenylalaninal), chymotrypsin inhibitor I, 3,4-dichloroisocoumarin, diisopropylfluoro phosphate, dipeptidylpeptidase IV inhibitor I (Ile-Pro-Ile), dipeptidylpeptidase IV inhibitor II (H-Glu-(NHO-Bz)-Pyr), ecotin, elastase inhibitor I (Boc-Ala-Ala-Ala-NHO-Bz), α2-macroglobulin, PPACK (D-Phe-Pro-Arg-chloromethylketone), PPACK II, N^{a}-tosyl-Lys chloromethyl ketone, N^{a}-tosyl-Phe chloromethyl ketone, acetyl-pepstatin (Ac-Val-Val-(3S,4S)-Sta-Ala-(3S,4S)-Sta-OH), calpain inhibitor I (N-acetyl-Leu-Leu-norleucinal), calpain inhibitor II (N-acetyl-Leu-Leu-Met-CHO), amastatin ([(2S,2R)]-3-amino-2-hydroxy-5-methylhexanoyl]-Val-Val-Asp-OH), arphamenine A ((2R,5S)-5-amino-8-guanidino-4-oxo-2-phenylmethyl octanoic acid), arphamenine B ((2R,5S)-5-amino-8-guanidino-4-oxo-2-p-hydroxyphenyl methyloctanoic acid), benzamidine, bestatin ([(2S,2R)-3-amino-2-hydroxy-4-phenyl butanoyl]-L-Leucine), CA-074 ((L-3-trans-[propylcarbamoyl]oxirane-2-carbonyl)-L-isoleucyl-L-proline), CA-074-Me ((L-3-trans-[propylcarbamoyl]oxirane-2-carbonyl)-L-isoleucyl-L-proline-methylester), calpastatin, calpeptin (benzyloxycarbonylleucyl-norleucinal), carboxypeptidase inhibitor, cathepsin inhibitor I (Z-Phe-Gly-NHO-Bz), cathepsin inhibitor II (Z-Phe-Gly-NHO-Bz-pMe), cathepsin inhibitor III (Z-Phe-Gly-NHO-Bz-pOMe), cathepsin B inhibitor I (Z-Phe-Ala-CH₂F), cathepsin B inhibitor II (Ac-Leu-Val-lysinal), cathepsin L inhibitor I (Z-Phe-Phe-CH₂F), cathepsin L inhibitor II (Z-Phe-Tyr-CHO), cathepsin L inhibitor III (Z-Phe-Tyr-(t-Bu)-CHN₂), cathepsin L inhibitor IV (1-naphthalenesulfonyl-Ile-Trp-CHO), cathepsin L inhibitor V (Z-Phe-Tyr(OtBu)-COCHO), cathepsin L inhibitor VI (N-(4-biphenylacetyl)-S-methylcysteine-(D)-Arg-Phe-β-phenethylamide), cathepsin S inhibitor (Z-Phe-Leu-COCHO), cystatin, diprotin A (H-Ile-Pro-Ile-OH), E-64 (trans-epoxysuccinyl-L-leucylamido-(4-guanidino)butane), E-64 d (loxistatin, or (2S,3S)-trans-epoxysuccinyl-L-leucylamido-3-methylbutane ethyl ester), ebelactone A (3,11-dihydroxy-2,4,6,8,10,12-hexamethyl-9-oxo-6-tetradecenoic 1,3-lactone), ebelactone B (2-ethyl-3,11-dihydroxy-4,6,8,10,12-penta methyl-9-oxo-6-tetradecenoic 1,3-lactone), EDTA (ethylenediamine tetraacetic acid), EGTA (ethyleneglycol-bis(β-aminoethyl)-N,N,N′,N′-tetraacetic acid), elastase inhibitor II (MeOSuc-Ala-Ala-Pro-Ala-CMK), elastase inhibitor III (MeOSuc-Ala-Ala-Pro-Val-CMK), elastatinal (Leu-(Cap)-Gln-Ala-al or N—[(S)-1-carboxy-isopentyl)-carbamoyl-alpha-(2-iminohexahydro-4(S)-pyrimidyl]-L-glycyl-L-glutaminyl-L-alaninal), E-64 (trans-epoxysuccinyl-L-leucylamido-(4-guanidino)butane), E-64d (loxistatin, or (2S,3S)-trans-epoxysuccinyl-L-leucylamido-3-methylbutane ethyl ester), N-ethyl maleimide, GGACK (1,5-dansyl-L-glutamyl-L-glycyl-L-arginine chloro methyl ketone), galardin (N-[(2S)-(methoxycarbonylmethyl)-4-methylpentanoyl]-L-tryptophan-methyl amide), 2-guanidinoethylmercaptosuccinic acid, hirudin, HIV protease inhibitor (Ac-Leu-Val-phenylalaninal), leuhistin (((2R,3S)-3-amino-2-hydroxy-2-(1H-imidazol-4-ylmethyl)-5-methyl)-5-methylhexanoic acid), leupeptin (acetyl-leucyl-leucyl-arginal), NCO-700, PEFABLOC SC (4-(2-aminoethyl)-benzenesulfonyl fluoride), pepstatin (isovaleryl-Val-Val-4-amino-3-hydroxy-6-methylheptanoyl-Ala-4-amino-3-hydroxy-6-methylheptanoic acid), phebestin ((2S,3R)-3-amino-2-hydroxy-4-phenylbutanoyl-L-valyl-L-phenylalanine), PMSF (phenyl methyl sulfonyl fluoride), phosphoramidon (N-alpha-L-rhamnopyranosyloxy(hydroxyl phosphinyl)-L-Leucyl-L-tryptophan, plummer's inhibitor (D,L-2-mercaptomethyl-3-guanidino-ethylthiopropanoic acid), 1,10-phenanthroline, subtilisin inhibitor I (Boc-Ala-Ala-NHO-Bz), subtilisin inhibitor II (Z-Gly-Phe-NHO-Bz), subtilisin inhibitor III (Z-Gly-Phe-NHO-Bz-pOMe), subtilisin inhibitor IV (Boc-Pro-Phe-NHO-Bz-pCl), subtilisin inhibitor V (Boc-Ala-Pro-Phe-NHO-Bz), TIMP-2 (tissue inhibitor of metalloproteinase 2), trypsin inhibitor, secretory leukocyte protease inhibitor, and any mixture there of.

29. The method of claim 27, wherein the agent that alters activities of G-protein coupled receptors and cAMP or pharmaceutically acceptable derivative is selected from the group consisting of AB-MECA (N⁶-4-aminobenzyl-5′-N-methylcarboxamidoadenosine), CPA (N⁶-cyclopentyladenosine), ADAC (N⁶-[4-[[[4-[[[(2-aminoethyl)amino]carbonyl]methyl]-anilino]carbonyl]methyl]phenyl]adenosine), CCPA (2-chloro-N⁶-cyclopentyl adenosine), CHA (N⁶-cyclohexyladenosine), GR79236 (N⁶-[1S,trans,2-hydroxycyclo pentyl]adenosine), S-ENBA ((2S)—N⁶-(2-endonorbanyl)adenosine), IAB-MECA (N-(4-amino-3-iodobenzyl)adenosine-5′-N-methylcarboxamidoadenosine), R—PIA (R—N⁶-(phenyl isopropyl)adenosine), ATL146e (4-{3-[6-amino-9-(5-ethylcarbamoyl-3,4-dihydroxy-tetrahydro-furan-2-yl)-9H-purin-2-yl]-prop-2-ynyl}-cyclohexanecarboxylic acid methyl ester), CGS-21680 (APEC or 2-[p-(2-carbonyl-ethyl)-phenyl ethyl amino]-5′-N-ethylcarboxamidoadenosine), CV1808 (2-phenylaminoadenosine), HENECA (2-hex-1-ynyl-5′-N-ethylcarboxamido adenosine), NECA (5′-N-ethyl-carboxamido adenosine), PAPA-APEC (2-(4-[2-[(4-aminophenyl)methylcarbonyl]ethyl]phenyl)ethylamino-5′-N-ethyl carboxamidoadenosine), DITC-APEC (2-[p-(4-isothiocyanatophenylamino thio carbonyl-2-ethyl)-phenylethylamino]-5′-N-ethylcarboxamidoadenosine), DPMA (N⁶-(2(3,5-dimethoxy phenyl)-2-(2-methylphenyl)ethyl)adenosine), S—PHPNECA ((S)-2-phenylhydroxypropynyl-5′-N-ethylcarboxamidoadenosine), WRC-0470 (2-cyclohexyl methylidenehydrazinoadenosine), AMP-579 (1S-[1a,2b,3b,4a(S*)]]-4-[7-[[2-(3-chloro-2-thienyl)-1-methylpropyl]amino]-3H-imidazo[4,5-b]pyridyl-3-yl]cyclopentane carboxamide), IB-MECA (N⁶-(3-iodobenzyl)adenosine-5′-N-methyluronamide), 2-CIADO (2-chloroadenosine), I-ABA (N⁶-(4-amino-3-iodobenzyl)adenosine), S—PIA (S—N⁶-(phenylisopropyl)adenosine), 2-[(2-aminoethyl-aminocarbonylethyl)phenylethyl amino]-5′-N-ethyl-carboxamidoadenosine, 2-Cl—IB-MECA (2-chloro-N⁶-(3-iodobenzyl)adenosine-5′-N-methyluronamide), adenosine, polyadenylic acid, and any mixture thereof.

30. A method of preventing organ or tissue injury at predetermined point or period of intervention comprising administrating to a living subject in need thereof a pharmaceutical composition comprising:
a. a protease inhibitor; and
b. an agent that alters activities of G protein coupled receptors and cAMP, an analog or a pharmaceutically acceptable derivative or prodrug thereof.

31. The method of claim 30, wherein the organ or tissue injury is related to at least one of cardiac surgery, non-surgical cardiac revascularization, organ transplantation, perfusion, ischemia, reperfusion, ischemia-reperfusion injury, oxidant injury, cytokine induced injury, shock induced injury, resuscitations injury, or apoptosis.

32. The method of claim 30, wherein the administration is made at the predetermined point of time related to at least one of pre-treatment regimen, pharmacological preconditioning, reperfusion or post interventional therapy, wherein the pharmacological preconditioning is a treatment administered before the ischemic intervention followed by a brief period of reperfusion or washout.

33. The method of claim 30, wherein the protease inhibitor is selected from the group consisting of 4-(2-aminoethyl)benzenesulfonylfluoride, ε-amino-n-caproic acid, α1-antichymotrypsin, antipain, antithrombin III, α1-antitrypsin, p-amidinophenylmethyl sulfonyl fluoride, aprotinin, cathepsin/subtilisin inhibitor (Boc-Val-Phe-NHO-Bz-pCl), chymostatin ([(S)-1-carboxy-2-phenylethyl]-carbamoyl-α-[2-amidohexahydro-4(S)-pyrimidyl]-(S)-glycyl-[A=Leu, B=Val, or C=Ile]-phenylalaninal), chymotrypsin inhibitor I, 3,4-dichloroisocoumarin, diisopropylfluoro phosphate, dipeptidylpeptidase IV inhibitor I (Ile-Pro-Ile), dipeptidylpeptidase IV inhibitor II (H-Glu-(NHO-Bz)-Pyr), ecotin, elastase inhibitor I (Boc-Ala-Ala-Ala-NHO-Bz), α2-macroglobulin PPACK (D-Phe-Pro-Arg-chloromethylketone), PPACK II, N^{a}-tosyl-Lys chloromethyl ketone, N^{a}-tosyl-Phe chloromethyl ketone, acetyl-pepstatin (Ac-Val-Val-(3S,4S)-Sta-Ala-(3S,4S)-Sta-OH), calpain inhibitor I (N-acetyl-Leu-Leu-norleucinal), calpain inhibitor II (N-acetyl-Leu-Leu-Met-CHO), amastatin ([(2S,2R)]-3-amino-2-hydroxy-5-methylhexanoyl]-Val-Val-Asp-OH), arphamenine A ((2R,5S)-5-amino-8-guanidino-4-oxo-2-phenylmethyl octanoic acid), arphamenine B ((2R,5S)-5-amino-8-guanidino-4-oxo-2-p-hydroxyphenyl methyloctanoic acid), benzamidine, bestatin ([(2S,2R)-3-amino-2-hydroxy-4-phenyl butanoyl]-L-Leucine), CA-074 ((L-3-trans-[propylcarbamoyl]oxirane-2-carbonyl)-L-isoleucyl-L-proline), CA-074-Me ((L-3-trans-[propylcarbamoyl]oxirane-2-carbonyl)-L-isoleucyl-L-proline-methylester), calpastatin, calpeptin (benzyloxycarbonylleucyl-norleucinal), carboxypeptidase inhibitor, cathepsin inhibitor I (Z-Phe-Gly-NHO-Bz), cathepsin inhibitor II (Z-Phe-Gly-NHO-Bz-pMe), cathepsin inhibitor III (Z-Phe-Gly-NHO-Bz-pOMe), cathepsin B inhibitor I (Z-Phe-Ala-CH₂F), cathepsin B inhibitor II (Ac-Leu-Val-lysinal), cathepsin L inhibitor I (Z-Phe-Phe-CH₂F), cathepsin L inhibitor II (Z-Phe-Tyr-CHO), cathepsin L inhibitor III (Z-Phe-Tyr-(t-Bu)-CHN₂), cathepsin L inhibitor IV (1-naphthalenesulfonyl-Ile-Trp-CHO), cathepsin L inhibitor V (Z-Phe-Tyr(OtBu)-COCHO), cathepsin L inhibitor VI (N-(4-biphenylacetyl)-S-methylcysteine-(D)-Arg-Phe-β-phenethylamide), cathepsin S inhibitor (Z-Phe-Leu-COCHO), cystatin, diprotin A (H-Ile-Pro-Ile-OH), E-64 (trans-epoxysuccinyl-L-leucylamido-(4-guanidino)butane), E-64 d (loxistatin, or (2S,3S)-trans-epoxysuccinyl-L-leucylamido-3-methylbutane ethyl ester), ebelactone A (3,11-dihydroxy-2,4,6,8,10,12-hexamethyl-9-oxo-6-tetradecenoic 1,3-lactone), ebelactone B (2-ethyl-3,11-dihydroxy-4,6,8,10,12-penta methyl-9-oxo-6-tetradecenoic 1,3-lactone), EDTA (ethylenediamine tetraacetic acid), EGTA aminoethyl)-N,N,N′,N′-tetraacetic acid), elastase inhibitor II (MeOSuc-Ala-Ala-Pro-Ala-CMK), elastase inhibitor III (MeOSuc-Ala-Ala-Pro-Val-CMK), elastatinal (Leu-(Cap)-Gln-Ala-al or N—[(S)-1-carboxy-isopentyl)-carbamoyl-alpha-(2-iminohexahydro-4(S)-pyrimidyl]-L-glycyl-L-glutaminyl-L-alaninal), E-64 (trans-epoxysuccinyl-L-leucylamido-(4-guanidino)butane), E-64d (loxistatin, or (2S,3S)-trans-epoxysuccinyl-L-leucylamido-3-methylbutane ethyl ester), N-ethyl maleimide, GGACK (1,5-dansyl-L-glutamyl-L-glycyl-L-arginine chloro methyl ketone), galardin (N-[(2S)-(methoxycarbonylmethyl)-4-methylpentanoyl]-L-tryptophan-methyl amide), 2-guanidinoethylmercaptosuccinic acid, hirudin, HIV protease inhibitor (Ac-Leu-Val-phenylalaninal), leuhistin (((2R,3S)-3-amino-2-hydroxy-2-(1H-imidazol-4-ylmethyl)-5-methyl)-5-methylhexanoic acid), leupeptin (acetyl-leucyl-leucyl-arginal), NCO-700, PEFABLOC SC (4-(2-aminoethyl)-benzenesulfonyl fluoride), pepstatin (isovaleryl-Val-Val-4-amino-3-hydroxy-6-methylheptanoyl-Ala-4-amino-3-hydroxy-6-methylheptanoic acid), phebestin ((2S,3R)-3-amino-2-hydroxy-4-phenylbutanoyl-L-valyl-L-phenylalanine), PMSF (phenyl methyl sulfonyl fluoride), phosphoramidon (N-alpha-L-rhamnopyranosyloxy(hydroxyl phosphinyl)-L-Leucyl-L-tryptophan, plummer's inhibitor (D,L-2-mercaptomethyl-3-guanidino-ethylthiopropanoic acid), 1,10-phenanthroline, subtilisin inhibitor I (Boc-Ala-Ala-NHO-Bz), subtilisin inhibitor II (Z-Gly-Phe-NHO-Bz), subtilisin inhibitor III (Z-Gly-Phe-NHO-Bz-pOMe), subtilisin inhibitor IV (Boc-Pro-Phe-NHO-Bz-pCl), subtilisin inhibitor V (Boc-Ala-Pro-Phe-NHO-Bz), TIMP-2 (tissue inhibitor of metalloproteinase 2), trypsin inhibitor, secretory leukocyte protease inhibitor, and any mixture there of.

34. The method of claim 30, wherein the agent that alters activities of G protein coupled receptors and cAMP is selected from the group consisting of AB-MECA (N⁶-4-amino benzyl-5′-N-methylcarboxamidoadenosine), CPA (N⁶-cyclopentyladenosine), ADAC (N⁶-[4-[[[4-[[[(2-aminoethyl)amino]carbonyl]methyl]-anilino]carbonyl]methyl]phenyl]adenosine), CCPA (2-chloro-N⁶-cyclopentyladenosine), CHA (N⁶-cyclohexyladenosine), GR79236 (N⁶-[1S,trans,2-hydroxycyclopentyl]adenosine), S-ENBA ((2S)—N⁶-(2-endonorbanyl)adenosine), IAB-MECA (N⁶-(4-amino-3-iodobenzyl)adenosine-5′-N-methylcarboxamidoadenosine), R—PIA (R—N⁶-(phenylisopropyl)adenosine), ATL146e (4-{3-[6-amino-9-(5-ethylcarbamoyl-3,4-dihydroxy-tetrahydro-furan-2-yl)-9H-purin-2-yl]-prop-2-ynyl}-cyclohexanecarboxylic acid methyl ester), CGS-21680 (APEC or 2-[p-(2-carbonyl-ethyl)-phenyl ethyl amino]-5′-N-ethylcarboxamidoadenosine), CV1808 (2-phenylaminoadenosine), HENECA (2-hex-1-ynyl-5′-N-ethylcarboxamido adenosine), NECA (5′-N-ethyl-carboxamido adenosine), PAPA-APEC (2-(4-[2-[(4-aminophenyl)methyl carbonyl]ethyl]phenyl)ethylamino-5′-N-ethyl carboxamidoadenosine), DITC-APEC (2-[p-(4-isothiocyanatophenylamino thiocarbonyl-2-ethyl)-phenylethylamino]-5′-N-ethylcarboxamidoadenosine), DPMA (N⁶-(2(3,5-dimethoxy phenyl)-2-(2-methyl phenyl)ethyl)adenosine), S—PHPNECA ((S)-2-phenylhydroxypropynyl-5′-N-ethylcarbox amidoadenosine), WRC-0470 (2-cyclohexylmethylidenehydrazinoadenosine), AMP-579 (1S-[1a,2b,3b,4a(S*)]]-4-[7-[[2-(3-chloro-2-thienyl)-1-methylpropyl]amino]-3H-imidazo[4,5-b]pyridyl-3-yl]cyclopentane carboxamide), IB-MECA (N⁶-(3-iodobenzyl)adenosine-5′-N-methyluronamide), 2-CIADO (2-chloroadenosine), I-ABA (N⁶-(4-amino-3-iodobenzyl)adenosine), S—PIA (S—N⁶-(phenylisopropyl)adenosine), 2-[(2-aminoethyl-aminocarbonylethyl)phenylethyl amino]-5′-N-ethyl-carboxamidoadenosine, 2-Cl—IB-MECA (2-chloro-N⁶-(3-iodobenzyl)adenosine-5′-N-methyluronamide), adenosine, polyadenylic acid, and any mixture thereof.
